SEQUENCE LISTING (132 sequences)

SEQ ID NO: 1 - protein, 28 aa, Artificial
  synthetic construct
GSSFLSPEHQRVQQRKESKKPPAKLQPR

SEQ ID NO: 2 - protein, 27 aa, Artificial
  synthetic construct
GSSFLSPEHQRVQRKESKKPPAKLQPR

SEQ ID NO: 3 - protein, 28 aa, Artificial
  synthetic construct
GSSFLSPEHQKAQQRKESKKPPAKLQPR

SEQ ID NO: 4 - protein, 28 aa, Artificial
  synthetic construct
RPQLKAPPKKSEKRQQVRQHEPSLFSSG

SEQ ID NO: 5 - protein, 28 aa, Artificial
  synthetic construct
GSXFLSPEHQRVQQRKESKKPPAKLQPR

SEQ ID NO: 6 - protein, 28 aa, Artificial
  synthetic construct
GSXFLSPEHQRVQQRKESKKPPAKLQPR

SEQ ID NO: 7 - protein, 30 aa, Artificial
  synthetic construct
GSSFLSPEHQRVQQRKESKKPPAKLQPRGG

SEQ ID NO: 8 - protein, 27 aa, Artificial
  synthetic construct
XSFLSPEHQRVQQRKESKKPPAKLQPR

SEQ ID NO: 9 - protein, 28 aa, Artificial
  synthetic construct
XSSFLSPEHQRVQQRKESKKPPAKLQPR

SEQ ID NO: 10 - protein, 5 aa, Artificial
  synthetic construct
LFSSG

SEQ ID NO: 11 - protein, 28 aa, Artificial
  synthetic construct
GSSFLSPEHQRVQQRKESKKPPAKLQPR

SEQ ID NO: 12 - protein, 28 aa, Artificial
  synthetic construct
GSSFLSPEHQRVQQRKESKKPPAKLQPR

SEQ ID NO: 13 - protein, 28 aa, Artificial
  synthetic construct
GSSFLSPEHQRVQQRKESKKPPAKLQPR

SEQ ID NO: 14 - protein, 7 aa, Artificial
  synthetic construct
KFWAXHA

SEQ ID NO: 15 - protein, 6 aa, Artificial
  synthetic construct
KXWAXA

SEQ ID NO: 16 - protein, 6 aa, Artificial
  synthetic construct
KFWAWH

SEQ ID NO: 17 - protein, 6 aa, Artificial
  synthetic construct
KFWAXH

SEQ ID NO: 18 - protein, 5 aa, Artificial
  Synthetic construct
KFXHX

SEQ ID NO: 19 - protein, 4 aa, Artificial
  Synthetic construct
SSFL

SEQ ID NO: 20 - protein, 28 aa, Artificial
  Synthetic construct
XSSFLSPEHQRVQQRKESKKPPAKLQPR

SEQ ID NO: 21 - protein, 27 aa, Artificial
  Synthetic construct
XSFLSPEHQRVQQRKESKKPPAKLQPR

SEQ ID NO: 22 - protein, 31 aa, Artificial
  Synthetic construct
XSFLSPEHQRVQQRKESKKPPAKLQPRGABA

SEQ ID NO: 23 - protein, 29 aa, Artificial
  Synthetic construct
XSSFLSPEHQRVQQRKESKKPPAKLQPRX

SEQ ID NO: 24 - protein, 27 aa, Artificial
  Synthetic construct
XSFLSPEHQRVQQRKESKKPPAKLQPR

SEQ ID NO: 25 - protein, 28 aa, Artificial
  Synthetic construct
XSSFLSPEHQRVQQRKESKKPPAKLQPR

SEQ ID NO: 26 - protein, 27 aa, Artificial
  Synthetic construct
XSFLSPEHQRVQQRKESKKPPAKLQPR

SEQ ID NO: 27 - protein, 28 aa, Artificial
  Synthetic construct
XSSFLSPEHQRVQQRKESKKPPAKLQPR

SEQ ID NO: 28 - protein, 27 aa, Artificial
  Synthetic construct
XSFLSPEHQRVQQRKESKKPPAKLQPR

SEQ ID NO: 29 - protein, 28 aa, Artificial
  Synthetic construct
XSSFLSPEHQRVQQRKESKKPPAKLQPR

SEQ ID NO: 30 - protein, 7 aa, Artificial
  Synthetic construct
FLSPEHQ

SEQ ID NO: 31 - protein, 6 aa, Artificial
  Synthetic construct
FLSPEH

SEQ ID NO: 32 - protein, 5 aa, Artificial
  Synthetic construct
FLSPE

SEQ ID NO: 33 - protein, 4 aa, Artificial
  Synthetic construct
FLSP

SEQ ID NO: 34 - protein, 3 aa, Artificial
  Synthetic construct
FLS

SEQ ID NO: 35 - protein, 25 aa, Artificial
  Synthetic construct
FLSPEHQRVQQRKESKKPPAKLQPR

SEQ ID NO: 36 - protein, 24 aa, Artificial
  Synthetic construct
FLSPEHQRVQQRKESKKPPAKLQP

SEQ ID NO: 37 - protein, 23 aa, Artificial
  Synthetic construct
FLSPEHQRVQQRKESKKPPAKLQ

SEQ ID NO: 38 - protein, 22 aa, Artificial
  Synthetic construct
FLSPEHQRVQQRKESKKPPAKL

SEQ ID NO: 39 - protein, 21 aa, Artificial
  Synthetic construct
FLSPEHQRVQQRKESKKPPAK

SEQ ID NO: 40 - protein, 20 aa, Artificial
  Synthetic construct
FLSPEHQRVQQRKESKKPPA

SEQ ID NO: 41 - protein, 19 aa, Artificial
  Synthetic construct
FLSPEHQRVQQRKESKKPP

SEQ ID NO: 42 - protein, 18 aa, Artificial
  Synthetic construct
FLSPEHQRVQQRKESKKP

SEQ ID NO: 43 - protein, 17 aa, Artificial
  Synthetic construct
FLSPEHQRVQQRKESKK

SEQ ID NO: 44 - protein, 16 aa, Artificial
  Synthetic construct
FLSPEHQRVQQRKESK

SEQ ID NO: 45 - protein, 15 aa, Artificial
  Synthetic construct
FLSPEHQRVQQRKES

SEQ ID NO: 46 - protein, 14 aa, Artificial
  Synthetic construct
FLSPEHQRVQQRKE

SEQ ID NO: 47 - protein, 13 aa, Artificial
  Synthetic construct
FLSPEHQRVQQRK

SEQ ID NO: 48 - protein, 12 aa, Artificial
  Synthetic construct
FLSPEHQRVQQR

SEQ ID NO: 49 - protein, 11 aa, Artificial
  Synthetic construct
FLSPEHQRVQQ

SEQ ID NO: 50 - protein, 10 aa, Artificial
  Synthetic construct
FLSPEHQRVQ

SEQ ID NO: 51 - protein, 9 aa, Artificial
  synthetic construct
FLSPEHQRV

SEQ ID NO: 52 - protein, 8 aa, Artificial
  synthetic construct
FLSPEHQR

SEQ ID NO: 53 - protein, 7 aa, Artificial
  synthetic construct
QHEPSLF

SEQ ID NO: 54 - protein, 6 aa, Artificial
  synthetic construct
HEPSLF

SEQ ID NO: 55 - protein, 5 aa, Artificial
  synthetic construct
EPSLF

SEQ ID NO: 56 - protein, 4 aa, Artificial
  synthetic construct
PSLF

SEQ ID NO: 57 - protein, 3 aa, Artificial
  synthetic construct
SLF

SEQ ID NO: 58 - protein, 25 aa, Artificial
  synthetic construct
RPQLKAPPKKSEKRQQVRQHEPSLF

SEQ ID NO: 59 - protein, 24 aa, Artificial
  synthetic construct
PQLKAPPKKSEKRQQVRQHEPSLF

SEQ ID NO: 60 - protein, 23 aa, Artificial
  synthetic construct
QLKAPPKKSEKRQQVRQHEPSLF

SEQ ID NO: 61 - protein, 22 aa, Artificial
  synthetic construct
LKAPPKKSEKRQQVRQHEPSLF

SEQ ID NO: 62 - protein, 21 aa, Artificial
  synthetic construct
KAPPKKSEKRQQVRQHEPSLF

SEQ ID NO: 63 - protein, 25 aa, Artificial
  synthetic construct
RPQLKAPPKKSEKRQQVRQHEPSLF

SEQ ID NO: 64 - protein, 24 aa, Artificial
  synthetic construct
PQLKAPPKKSEKRQQVRQHEPSLF

SEQ ID NO: 65 - protein, 23 aa, Artificial
  synthetic construct
QLKAPPKKSEKRQQVRQHEPSLF

SEQ ID NO: 66 - protein, 22 aa, Artificial
  synthetic construct
LKAPPKKSEKRQQVRQHEPSLF

SEQ ID NO: 67 - protein, 21 aa, Artificial
  synthetic construct
KAPPKKSEKRQQVRQHEPSLF

SEQ ID NO: 68 - protein, 20 aa, Artificial
  synthetic construct
APPKKSEKRQQVRQHEPSLF

SEQ ID NO: 69 - protein, 19 aa, Artificial
  synthetic construct
PPKKSEKRQQVRQHEPSLF

SEQ ID NO: 70 - protein, 18 aa, Artificial
  synthetic construct
PKKSEKRQQVRQHEPSLF

SEQ ID NO: 71 - protein, 17 aa, Artificial
  synthetic construct
KKSEKRQQVRQHEPSLF

SEQ ID NO: 72 - protein, 16 aa, Artificial
  synthetic construct
KSEKRQQVRQHEPSLF

SEQ ID NO: 73 - protein, 15 aa, Artificial
  synthetic construct
SEKRQQVRQHEPSLF

SEQ ID NO: 74 - protein, 14 aa, Artificial
  synthetic construct
EKRQQVRQHEPSLF

SEQ ID NO: 75 - protein, 13 aa, Artificial
  synthetic construct
KRQQVRQHEPSLF

SEQ ID NO: 76 - protein, 12 aa, Artificial
  synthetic construct
RQQVRQHEPSLF

SEQ ID NO: 77 - protein, 11 aa, Artificial
  synthetic construct
QQVRQHEPSLF

SEQ ID NO: 78 - protein, 10 aa, Artificial
  synthetic construct
QVRQHEPSLF

SEQ ID NO: 79 - protein, 9 aa, Artificial
  synthetic construct
VRQHEPSLF

SEQ ID NO: 80 - protein, 8 aa, Artificial
  synthetic construct
RQHEPSLF

SEQ ID NO: 81 - protein, 31 aa, Artificial
  synthetic construct
GSSXFLSPEHQRVQQRKESKKPPAKLQPRXX

SEQ ID NO: 82 - protein, 25 aa, Artificial
  synthetic construct
FLSPEHQKVQQRKESKKPPAKLQPR

SEQ ID NO: 83 - protein, 24 aa, Artificial
  synthetic construct
FLSPEHQKVQQRKESKKPPAKLQP

SEQ ID NO: 84 - protein, 23 aa, Artificial
  synthetic construct
FLSPEHQKVQQRKESKKPPAKLQ

SEQ ID NO: 85 - protein, 22 aa, Artificial
  synthetic construct
FLSPEHQKVQQRKESKKPPAKL

SEQ ID NO: 86 - protein, 21 aa, Artificial
  synthetic construct
FLSPEHQKVQQRKESKKPPAK

SEQ ID NO: 87 - protein, 20 aa, Artificial
  synthetic construct
FLSPEHQKVQQRKESKKPPA

SEQ ID NO: 88 - protein, 19 aa, Artificial
  synthetic construct
FLSPEHQKVQQRKESKKPP

SEQ ID NO: 89 - protein, 18 aa, Artificial
  synthetic construct
FLSPEHQKVQQRKESKKP

SEQ ID NO: 90 - protein, 17 aa, Artificial
  synthetic construct
FLSPEHQKVQQRKESKK

SEQ ID NO: 91 - protein, 16 aa, Artificial
  synthetic construct
FLSPEHQKVQQRKESK

SEQ ID NO: 92 - protein, 15 aa, Artificial
  synthetic construct
FLSPEHQKVQQRKES

SEQ ID NO: 93 - protein, 14 aa, Artificial
  synthetic construct
FLSPEHQKVQQRKE

SEQ ID NO: 94 - protein, 13 aa, Artificial
  synthetic construct
FLSPEHQKVQQRK

SEQ ID NO: 95 - protein, 12 aa, Artificial
  synthetic construct
FLSPEHQKVQQR

SEQ ID NO: 96 - protein, 11 aa, Artificial
  synthetic construct
FLSPEHQKVQQ

SEQ ID NO: 97 - protein, 10 aa, Artificial
  synthetic construct
FLSPEHQKVQ

SEQ ID NO: 98 - protein, 9 aa, Artificial
  synthetic construct
FLSPEHQKV

SEQ ID NO: 99 - protein, 8 aa, Artificial
  synthetic construct
FLSPEHQK

SEQ ID NO: 100 - protein, 25 aa, Artificial
  synthetic construct
FLSPEHQRAQQRKESKKPPAKLQPR

SEQ ID NO: 101 - protein, 24 aa, Artificial
  synthetic construct
FLSPEHQRAQQRKESKKPPAKLQP

SEQ ID NO: 102 - protein, 23 aa, Artificial
  synthetic construct
FLSPEHQRAQQRKESKKPPAKLQ

SEQ ID NO: 103 - protein, 22 aa, Artificial
  synthetic construct
FLSPEHQRAQQRKESKKPPAKL

SEQ ID NO: 104 - protein, 21 aa, Artificial
  synthetic construct
FLSPEHQRAQQRKESKKPPAK

SEQ ID NO: 105 - protein, 20 aa, Artificial
  synthetic construct
FLSPEHQRAQQRKESKKPPA

SEQ ID NO: 106 - protein, 19 aa, Artificial
  synthetic construct
FLSPEHQRAQQRKESKKPP

SEQ ID NO: 107 - protein, 18 aa, Artificial
  synthetic construct
FLSPEHQRAQQRKESKKP

SEQ ID NO: 108 - protein, 17 aa, Artificial
  synthetic construct
FLSPEHQRAQQRKESKK

SEQ ID NO: 109 - protein, 16 aa, Artificial
  synthetic construct
FLSPEHQRAQQRKESK

SEQ ID NO: 110 - protein, 15 aa, Artificial
  synthetic construct
FLSPEHQRAQQRKES

SEQ ID NO: 111 - protein, 14 aa, Artificial
  synthetic construct
FLSPEHQRAQQRKE

SEQ ID NO: 112 - protein, 13 aa, Artificial
  synthetic construct
FLSPEHQRAQQRK

SEQ ID NO: 113 - protein, 12 aa, Artificial
  synthetic construct
FLSPEHQRAQQR

SEQ ID NO: 114 - protein, 11 aa, Artificial
  synthetic construct
FLSPEHQRAQQ

SEQ ID NO: 115 - protein, 10 aa, Artificial
  synthetic construct
FLSPEHQRAQ

SEQ ID NO: 116 - protein, 9 aa, Artificial
  synthetic construct
FLSPEHQRA

SEQ ID NO: 117 - protein, 25 aa, Artificial
  synthetic construct
FLSPEHQKAQQRKESKKPPAKLQPR

SEQ ID NO: 118 - protein, 24 aa, Artificial
  synthetic construct
FLSPEHQKAQQRKESKKPPAKLQP

SEQ ID NO: 119 - protein, 23 aa, Artificial
  synthetic construct
FLSPEHQKAQQRKESKKPPAKLQ

SEQ ID NO: 120 - protein, 22 aa, Artificial
  synthetic construct
FLSPEHQKAQQRKESKKPPAKL

SEQ ID NO: 121 - protein, 21 aa, Artificial
  synthetic construct
FLSPEHQKAQQRKESKKPPAK

SEQ ID NO: 122 - protein, 20 aa, Artificial
  synthetic construct
FLSPEHQKAQQRKESKKPPA

SEQ ID NO: 123 - protein, 19 aa, Artificial
  synthetic construct
FLSPEHQKAQQRKESKKPP

SEQ ID NO: 124 - protein, 18 aa, Artificial
  synthetic construct
FLSPEHQKAQQRKESKKP

SEQ ID NO: 125 - protein, 17 aa, Artificial
  synthetic construct
FLSPEHQKAQQRKESKK

SEQ ID NO: 126 - protein, 16 aa, Artificial
  synthetic construct
FLSPEHQKAQQRKESK

SEQ ID NO: 127 - protein, 15 aa, Artificial
  synthetic construct
FLSPEHQKAQQRKES

SEQ ID NO: 128 - protein, 14 aa, Artificial
  synthetic construct
FLSPEHQKAQQRKE

SEQ ID NO: 129 - protein, 13 aa, Artificial
  synthetic construct
FLSPEHQKAQQRK

SEQ ID NO: 130 - protein, 12 aa, Artificial
  synthetic construct
FLSPEHQKAQQR

SEQ ID NO: 131 - protein, 11 aa, Artificial
  synthetic construct
FLSPEHQKAQQ

SEQ ID NO: 132 - protein, 10 aa, Artificial
  synthetic construct
FLSPEHQKAQ

(19) United States
(12) Patent Application Publication
(10) Pub. No.: US 2008/0300180 A1
(43) Pub. Date: Dec. 4, 2008

(54) Growth Hormone Secretagogue Receptor 1A Ligands

(75) Inventors: Hans T. Schambye, Holte (DK); Birgitte Holst Lange, Gentofte (DK); Peter Holme Jensen, Copenhagen (DK)

Correspondence Address: BROWDY AND NEIMARK, P.L.L.C.;624 NINTH STREET, NW, SUITE 300, WASHINGTON, DC, 20001-5303, US

(73) Assignee: Gastrotech Pharma A/S, Copenhagen K (DK)

(21) Appl. No.: 11/720,477

(22) PCT Filed: Nov. 29, 2005
(86) PCT No.: PCT/DK05/00763

§ 371 Date: May 30, 2007

(30) Foreign Application Priority Data
Nov. 30, 2004 (DK) PA2004 01875

(51) Int. Cl. A61K 9/127; A61K 38/18; A61K 38/05; A61K 38/06; A61K 38/07; A61K 38/08; A61K 38/10; C07K 7/00; C07K 5/00; C07K 14/475; A61P 3/00

(52) U.S. Cl. 514/12; 530/331; 530/330; 530/332; 530/329; 530/325; 530/324; 530/326; 530/327; 530/328; 514/13; 514/14; 514/15; 514/16; 514/17; 514/18; 514/19

(57) ABSTRACT

The present invention relates to new growth hormone secretagogue receptor 1A (GHS-R 1A) ligands, and pharmaceutical compositions comprising any of the new GHS-R1 A ligands. The ligands are suitable for a wide range of applications, and thus the present invention also relates to use of the GHS-R1 A ligands according to the present invention in the manufacture of a medicament for the treatment of an individual in need thereof. In another aspect, the present invention relates to a method of treatment of an individual in need thereof, comprising administering to said individual one or more of the GHS-R1A ligands disclosed herein, such as e.g. for treatment of cancer cachexia.

This application claims priority from Danish patent application number PA 2004 01875, filed 30 Nov. 2004, which is hereby incorporated by reference in its entirety. All patent and non-patent references cited in this application and the present application, are also hereby incorporated by reference in their entirety.

### FIELD OF INVENTION

The present invention relates to new growth hormone secretagogue receptor 1A (GHS-R 1A) ligands, suitable for a wide range of applications, and pharmaceutical compositions comprising any of the new GHS-R1A ligands.

### BACKGROUND OF INVENTION

### Ghrelin

Ghrelin is a 28 amino acid peptide hormone primarily secreted into the circulation from the stomach but also synthesized in a number of peripheral tissues and in brain areas suggesting a role as both an endocrine and a paracrine hormone and a neurotransmitter. Ghrelin has a unique chemical structure among peptide hormones as it is acylated in a serine in position 3. The acylation appears to be crucial for binding and activation of ghrelin to its receptor, the growth hormone secretagogue (GHS) receptor 1a (GHS-R1a). GHS-R1a belongs to the large family of 7TM receptors, which most often signals through coupling to G-protein (Kojima M et al., Ghrelin: discovery of the natural endogenous ligand for the growth hormone secretagogue receptor. Trends Endocrinol. Metab 2001; 12(3):118-22.).

Ghrelin is secreted in the pre-meal situation which results in a surge in plasma levels that starts approximately 1-2 hour before a meal is initiated. The plasma ghrelin level decreases shortly after initiation of the meal (Cummings D E, et al., A preprandial rise in plasma ghrelin levels suggests a role in meal initiation in humans. Diabetes 2001; 50(8):1714-9). Since this is the only known endogenous peripherally produced orexigenic substance, it is believed that the increase in plasma level of ghrelin is of crucial importance for the initiation of a meal.

Pharmacological doses of ghrelin have a strong acute stimulatory effect on growth hormone secretion, but it does not seem to affect the endogenous growth hormone secretion on a chronic basis sufficiently to provide an alternative to growth hormone administration. It has been reported that some degree of overlap between the ghrelin and GH release during fasting and sleep, respectively, exists (Muller A F, et al. Ghrelin drives GH secretion during fasting in man. Eur. J. Endocrinol. 2002; 146(2):203-7; Koutkia P, et al. Nocturnal ghrelin pulsatility and response to growth hormone secretagogues in healthy men. Am. J. Physiol Endocrinol. Metab 2004; 287(3):E506-E512), whereas this is not the case during exercise or insulin induced low blood glucose (Broglio F, et al., Ghrelin does not mediate the somatotroph and corticotroph responses to the stimulatory effect of glucagon or insulin-induced hypoglycaemia in humans. Clin Endocrinol (Oxf) 2004; 60(6):699-704; Dall R, et al., Plasma ghrelin levels during exercise in healthy subjects and in growth hormone-deficient patients. Eur. J. Endocrinol. 2002; 147(1):65-70).

### Ghrelin Analogues

Analogues of ghrelin have been described in prior art, for example:
WO 200432952: Use of ghrelin for treatment of malnutrition in gastrectomized individuals
WO 2004009616: Synthesis and therapeutic uses of ghrelin analogs
WO 2001092292: Ghrelin analogs for use in screening compounds with growth hormone secretagogue receptor-activating ability and for inducing growth hormone secretion
WO 2001007475: Novel ghrelins, their encoding DNA sequences, and their use as therapeutics
Bednarek M A et al., “Structure-function studies on the new growth hormone-releasing peptide, ghrelin: minimal sequence of ghrelin necessary for activation of growth hormone secretagogue receptor 1a”, J Med. Chem. 2000 Nov. 16; 43(23):4370-6.

### SUMMARY OF INVENTION

The present invention relates to new growth hormone secretagogue receptor 1A (GHS-R 1A) ligands, suitable for a wide range of applications, and pharmaceutical compositions comprising any of the new GHS-R1A ligands. The present invention also relates to use of the GHS-R1A ligands according to the present invention in the manufacture of a medicament for the treatment of an individual in need thereof, and in methods of treatment.

Natural ghrelin has an anchor group comprising an acylated serine residue, which aids anchorage in the cell membrane. The first aspect of the present invention relates to novel GHS-R1a ligands comprising improved anchor groups. Without being bound by theory, it is contemplated that these anchor groups improve the anchorage of the GHS-R1A ligand in the cell membrane and thus improve the efficacy of the GHS-R1A ligand.

Thus, the first aspect of the present invention relates to a GHS-R1A ligand, or pharmaceutically acceptable salt thereof, wherein the GHS-R1A ligand is defined by formula I

Z¹-(X¹)ₘ-(X²)-(X³)ₙ-Z² Formula I
wherein
Z¹ is an optionally present protecting group;
each X¹ is independently selected from an amino acid, wherein said amino acid is selected from naturally occurring and synthetic amino acids;
and wherein X² is an anchor group with the proviso that when (X¹)ₘ-(X²)-(X³)ₙ has the amino acid sequence of any wildtype ghrelin, such as human or rat, the anchor group is different from acylated serine.
each X³ is independently selected from an amino acid, wherein said amino acid is selected from naturally occurring and synthetic amino acids,
Z² is an optionally present protecting group,
m is 0 or an integer in the range of 1-10
n is 0 or an integer in the range of 1-35;
   wherein m and n cannot both be 0.
The anchor group may be a lipid anchor group. Preferred anchor groups can be either:
any amino acid selected from naturally occurring and synthetic amino acids, said amino acid being modified with a group selected from:
a) a glycerophospholipid
b) a sterol moiety
c) a sphingolipid moiety
d) a ceramide or an analogue thereof
e) an isoprenoid pyrophosphate
f) a glycosyl-phosphatidylinositol (GPI) anchor
g) a phosphatidyl serine, or analogue thereof;
   or alternatively X² can be selected from:
h) decenoic acid (L or D form);
i) Trp(5-NH2) (L or D form);
j) 5-hexenoic acid (L or D form)
k) 6-heptenoic acid (L or D form)
l) 7-octenoic acid (L or D form)
m) 8-nonenoic acid (L or D form)
n) Ala-3-cp (L or D form)
o) Ala-3-cb (L or D form)
p) Phe-4-Me (L or D form)
q) Phe-4-Et (L or D form)
r) Phe-4-iPr (L or D form)
s) Phe-4-Ph (L or D form)
t) Beta-MeTrp (L or D form)
u) Ala[3-(3-Quinolinyl)] (L or D form)
v) Ala[3-(2-benzimidazoyl)] (L or D form)
w) BenzoTrp (L or D form)
x) 7-AzaTrp (L or D form);

In a second aspect of the invention a GHS-R1A ligand or a pharmaceutically acceptable salt thereof is provided,
wherein the GHS-R1A ligand has a structure defined by formula I′

Z²-(X³)ₙ-(X²)-(X¹)ₘ-Z³-Z¹ Formula I′
wherein Z¹ is an optionally present protecting group,
each X¹ is an amino acid independently selected from naturally occurring and synthetic amino acids,
X² is an anchor group, preferably selected from naturally occurring and synthetic amino acids modified with a bulky group, more preferably a modified D-amino acid,
each X³ is independently selected from an amino acid, wherein said amino acid is selected from naturally occurring and synthetic amino acids, with the proviso that at least one (X³) is a D-amino acid,
Z² is an optionally present protecting group,
Z³ is an optionally present linker or C-terminal group,
m is 0 or an integer in the range of 1-3
n is 0 or an integer in the range of 1-35,
and wherein both n and m cannot be 0.

In a third aspect of the present invention, a GHS-R1a ligand, or a pharmaceutically acceptable salt thereof, is provided,
wherein the GHS-R1A ligand has a structure defined by formula I″:

Z¹-X¹-X²-X³-X⁴-X⁵-X⁶-Z², Formula I″
wherein
Z¹ is an optionally present protecting group;
X¹ is an amino acid having a structure defined by motif A:

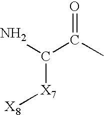

X², X³ and X⁵ are aromatic amino acids independently selected from naturally occurring and synthetic amino acids,
X⁴ is an optionally present amino acid selected from naturally occurring and synthetic amino acids,
and wherein optionally at least one of X², X³, X⁴ and X⁵ is an anchor group,
X⁶ is optionally present selected from the group consisting of:
a) an alcohol
b) an ether
c) a hydrocarbon
d) a hydrazine
e) a peptide
f) a peptidomimetic moiety;
X₇ is a spacer with length of 1-8 chemical bonds
X₈ is a hydrogen bond donor, such as an amine or hydroxyl group;
and Z² is an optionally present protecting group,
with the proviso that at least one of X¹-X⁵ is a D-amino acid.

The fourth aspect of the present invention relates to improved N-terminally-modified GHS-R1A ligands. Without being bound by theory, it is contemplated that the N-terminal modifications improve stability and effectiveness of the GHS-R1A ligands. Thus, in the fourth aspect of the present invention, a GHS-R1A ligand, or a pharmaceutically acceptable salt thereof, is provided,
wherein the GHS-R1A ligand has a structure defined by formula I′″

Z¹-R¹-(X²)-(X³)ₙ-Z² Formula I′″

wherein
Z¹ is an optionally present protecting group;
R¹ is selected from:
a) βAla- or
b) βAla-X¹- or
c) GABA- or
d) GABA-X¹- or
e) Aminopentanoyl-X¹- or
f) hydroxy acetic acid (HAA)- or
g) HM-X1- or
h) a compound with formula B, shown below:

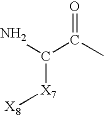

i) a compound with the formula i) shown below:
j) a compound with the formula j) shown below:

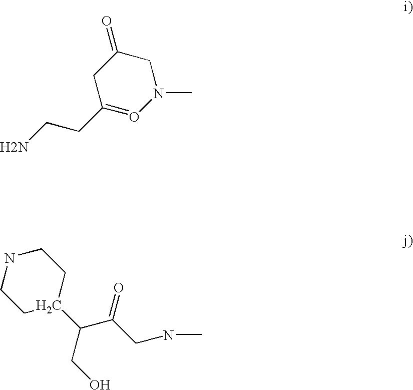

wherein X7 is a spacer with a length of 1-8 chemical bonds, and X8 is a hydrogen bond donor, such as an amine or hydroxyl group;
X¹ is an amino acid selected from naturally occurring and synthetic amino acids;
X² is any amino acid selected from naturally occurring and synthetic amino acids, said amino acid being modified with an anchor group;
each X³ is independently selected from an amino acid, wherein said amino acid is selected from naturally occurring and synthetic amino acids,
Z² is an optionally present protecting group,
n is 0 or an integer in the range of 1-35.

For example, the compound may have sequence according to any of SEQ ID NO: 20-29, or an analogue or homologue thereof.

In the fifth aspect of the present invention, a GHS-R1A ligand is provided with the following structure:

(SEQ ID NO: 81)

GSS(X²)FLSPEHQRVQQRKESKKPPAKLQPRXX,

wherein “XX” represents two amino acid moieties, each of which is independently selected from natural and synthetic amino acid moieties, and X² represents any of the anchor groups described herein, preferably (CO-C7 H15). In one preferred embodiment, said ligand has the sequence according to SEQ ID NO: 7.

The GHS-R1a ligands of the present invention may be GHS-R1a agonists, GHS-R1a partial agonists or GHS-R1a antagonists.

In another aspect, the present invention also relates to a pharmaceutical composition comprising one, two or more of any the GHS-R1A ligands described herein, or pharmaceutically acceptable salt(s) thereof, optionally further comprising another type of GHS-R1a ligand, such as wildtype ghrelin or any analogue thereof known in the art. In another aspect, the present invention also relates to use of one or more of the GHS-R1A ligands according to the present invention in the manufacture of a medicament for the treatment of an individual in need thereof. It is preferred that the individual is suffering from, or at risk of suffering from, a pathological condition treatable with a GHS-R1a ligand (such as ghrelin). In another aspect, the present invention relates to a method of treatment of an individual in need thereof, comprising administering to said individual one or more of the GHS-R1A ligands disclosed herein.

### DETAILED DESCRIPTION OF THE INVENTION

### Definitions

Alcohols or modified alcohols: Compounds in which a hydroxy group, —OH, is attached to a saturated carbon atom: R₃COH. Preferred alcohols for use in the present invention include, but are not restricted to, methanol, ethanol, isopropyl alcohol ethylene glycol, glycerol and phenol. The term “alcohol” in one embodiment includes fatty alcohols, including:

erucyl alcohol
ricinolyl alcohol
arachidyl alcohol
capryl alcohol
capric alcohol
behenyl alcohol
lauryl alcohol (1-dodecanol)
myristyl alcohol (1-tetradecanol)
cetyl (or palmityl) alcohol (1-hexadecanol)
stearyl alcohol (1-octadecanol)
isostearyl alcohol
oleyl alcohol (cis-9-octadecen-1-ol)
linoleyl alcohol (9Z,12Z-octadecadien-1-ol)
elaidolinoleyl alcohol (9E,12E-octadecadien-1-ol)
linolenyl alcohol (9Z,12Z,15Z-octadecatrien-1-ol)
elaidolinolenyl alcohol (9E,12E, 15-E-octadecatrien-1-ol)

Ether: Compounds with the formula ROR (wherein R is not equal to H). Preferably refers to any of a class of organic compounds in which two hydrocarbon groups are linked by an oxygen atom.

Preferred ethers for use in the present invention include, but are not restricted to, CH₃CH₂OCH₂CH₃ or any of the two structures shown below:

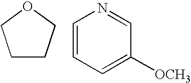

Hydrocarbons or substituted hydrocarbons: a hydrocarbon is an organic compound consisting of a carbon backbone with atoms of hydrogen attached to that backbone. Preferred hydrocarbons for use in the present invention are in one embodiment saturated hydrocarbons, which do not have double, triple or aromatic bonds between the carbon atoms. In another embodiment of the present invention, preferred hydrocarbons are unsaturated hydrocarbons, which have one or more double or triple bonds between carbon atoms. Preferred unsaturated hydrocarbons are:

alkenes—hydrocarbons that have a double bond between two carbon atoms.
alkynes—hydrocarbons that have at least one triple bond between carbon atoms.
dienes—hydrocarbons which comprise two double bonds.

Preferred hydrocarbons for use in the present invention include, but are not restricted to:

a) Alkyl group
b) Alkenyl group
c) Alkynyl group
d) Aryl group
e) Heterocyclyl group
f) Heteroaryl group
g) Cycloalkyl group
a) Alkyl group: the term “alkyl group” means a saturated linear or branched hydro-carbon group including, for example, methyl, ethyl, isopropyl, t-butyl, heptyl, dodecyl, octadecyl, amyl, 2-ethylhexyl, and the like. Preferred alkyls are lower alkyls, i.e. alkyls having 1 to 6 carbon atoms, such as 1, 2, 3, 4, 5 or 6 carbon atoms. Preferred alkyl groups include substituted lower alkyls having one to three substituents selected from the group consisting of hydroxyl, alkoxy, amino, amido, carboxyl, acyl, halogen, cyano, nitro and thiol.
b) Alkenyl group: the term “alkenyl” means a non-saturated linear or branched hydrocarbon group including, for example, methylene or ethylene.
c) Alkynyl group: the term “alkynyl” means a non-saturated linear or branched hydro-carbon group including, for example, ethynyl or propynyl.
d) Aryl represents a hydrocarbon comprising at least one aromatic ring, and may contain from 5 to 18, preferably from 6 to 14, more preferably from 6 to 10, and most preferably 6 carbon atoms. Typical aryl groups include phenyl, naphthyl, phenanthryl, anthracyl, indenyl, azulenyl, biphenylenyl, and fluorenyl groups. Particularly preferred aryl groups include phenyl, naphthyl and fluorenyl, with phenyl being most preferable.
e) Heterocyclyl means a monovalent saturated cyclic radical, consisting of one to two rings, of three to eight atoms per ring, incorporating one or two ring heteroatoms, chosen from N, O or S(O)₀₋₂, and which can optionally be substituted with one or two substituents selected from the group consisting of hydroxyl, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminofarbonyl, arylaminocarbonyl, alkylcarbonylamino, or arylcarbonylamino.
f) Heteroaryl means a monovalent aromatic cyclic radical having one to three rings, of four to eight atoms per ring, incorporating one or two heteroatoms (chosen from nitrogen, oxygen, or sulfur) within the ring which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonlamino and arylcarbonylamino.
g) Cycloalkyl means a monovalent saturated carbocyclic radical consisting of one or two rings, of three to eight carbons per ring, which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino and arylcarbonylamino.

A hydrocarbon for use in the present invention may also optionally further substituted one or more times with C, S, N, O, OH, phenyl, amine (NH), halogen, substituted lower alkyl, aryl, heterocyclyl, heteroaryl, aryl-(C1-4)-alkyl, heteroaryl-(C1-4)-alkyl, heterocyclyl-(C1-4)-alkyl, cycloalkylalkyl, cycloalkyl, alkoxy, carboxy, halogen, trifluoromethyl, cyano, amino, or nitro group.

Hydrazines—compounds with chemical formula N₂H₄ and derivatives thereof.

Peptide: same meaning as “polypeptide” (see below)

Peptidomimetic moiety: A compound that mimics the biological action of a peptide. Preferred peptidomimetic moieties for use in the present invention include, but are not restricted to, peptoids, reduced peptide bonds, PNA, LNA, D-amino acids and unnatural amino acids. Most preferred are peptoids and reduced peptide bonds.

Aromatic moiety: the term “aromatic” or “aryl” moiety means either a mono- or polycyclic hydrocarbon group, which has a cyclic, delocalized (4n+2) pi-electron system, including arenes and their substitution products. Examples of suitable aromatic moieties for use in the present invention include, but are not restricted to, benzene, naphthalene, toluene, thiophene and pyridine

Hydrophobic moiety or hydrophobic section: lipophilic species, preferably electrically neutral and nonpolar, preferring other neutral and nonpolar solvents or molecular environments. Suitable hydrophobic moieties for use in the present invention include, but are not restricted to, alkanes, norleucine, tryptophan, leucine, phenylalanine, valine, homoleucine, homoisoleucine, napthyl alanine and cyclohexylalanine. A moiety or section of a molecule may e.g. be considered “hydrophobic” using the Kow scale, wherein Kow=Concentration in octanol phase/Concentration in aqueous phase. A “hydrophobic” moiety or section of a molecule should have a Kow greater than than 100, such as greater than 200, for example greater than 300, such as greater than 400, for example greater than 500, such as greater than 600, for example greater than 700, such as greater than 800, for example greater than 900, such as 950-1000. Alternatively, log Kow should be 2-3, such as 2.1-3, for example 2.2-3, such as 2.3-3, for example 2.4-3, such as 2.5-3, for example 2.6-3, such as 2.7-3, for example 2.8-3, such as 2.9-3. Most preferably, the hydrophobic section considered is capable of binding to a phosphlipid membrane.

Amphiphilic moiety: a moiety containing both polar, water-soluble and nonpolar, water-insoluble groups. Examples of amphiphilic moieties suitable for use in the present invention include, but are not restricted to, phosphatidyl serine, ceramides, sphingolipids and isoprenoide pyrophosphates.

Affinity: the strength of binding between receptors and their ligands (for example between the GHS-receptor 1a and a ligand according to the present invention) and may be expressed as dissociation constant (kd) or inhibition constant (ki).

Amino Acid Residue: An amino acid formed upon chemical digestion (hydrolysis) of a polypeptide at its peptide linkages. The term “amino acid” encompasses every amino acid such as L-amino acid, D-amino acid, alpha-amino acid, beta-amino acid, gamma-amino acid, natural amino acid and synthetic amino acid or the like as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left-to-right orientation in the conventional direction of amino terminus to carboxy terminus. In addition, the phrase “amino acid residue” is broadly defined to include the amino acids listed in the Table of Correspondence and modified and non-naturally occurring amino acids. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Anti-neoplastic treatment: Treatment aimed at halting or reducing abnormal tissue growth (such as a neoplasm) in an individual. Examples of such treatment include cancer therapies, such as radiotherapy or chemotherapy.

Appetite: Appetite in an individual is assessed by measuring the amount of food ingested and by assessing the individual's desire to eat. Appetite (e.g. hunger) is typically assessed with a short questionnaire given to individuals on a random basis several times a week. Typically, subjects rate their hunger, preoccupation with food, and desire to eat greater quantities and different types of food by answering the questions using analogue scales ranging from 1, not at all, to 10, extremely.

BMI measures your height/weight ratio. It is determined by calculating weight in kilograms divided by the square of height in meters. The BMI “normal” range is 19-25, preferably an individual has a BMA of 19-22

Body fat mass: Body fat mass can be measured e.g. by the fat fold technique: In this technique, a pincer-type caliper is used to measure subcutaneous fat by determining skin fold thickness at representative sites on the body. These skin fold measurements are then used to compute body fat by either adding the scores from the various measurements and using this value as an indication of the relative degree of fatness among individuals or by using the measurements in mathematical equations that have been developed to predict percent body fat. Body composition can also be assessed by Dual Energy X-ray Absorptiometry (DEXA) scanning, a non-invasive test which accurately quantifies the lean body mass, the total fat mass and regional body fat (e.g. abdominal fat).

Concentration equivalent: A concentration equivalent is an Equivalents dosage being defined as the dosage of a GHS-R1A ligand having in vitro and/or in vivo the same response as evaluated from a dosage-response curve of wild-type ghrelin.

Dissociation constant, Kd: a measure to describe the strength of binding (or affinity or avidity) between receptors and their ligands. Most often Kd is measured by use of a radiolabled ligand. The smaller the Kd the stronger the binding.

Inhibition constant, ki: a measure to describe the strength of binding (or affinity or avidity) between receptors and their ligands. Where the ligand (L) of interest is not radiolabled, Ki describes the ability of L to displace the radioligand.

Euthyroid state: an individual is defined as having a euthyroid state if they are neither hyperthyroidic nor hypothyroidic.

Ghrelin: a polypeptide as described in Kojima M et al.; “Ghrelin is a growth hormone-releasing acylated peptide from stomach. Nature 402:656-660, 1999). Human 28 aa ghrelin has the amino acid sequence of SEQ ID NO: 1.

GHS: growth hormone secretagogue

GHS-R1a: the receptor for GHS. GHS-R1a is also denoted GHS 1a. The receptor has GENBANK accession number NM_{—}198407

Hydrogen bond donor: a strongly electronegative heteroatom attached to a hydrogen atom, such as NH₂ or OH.

HAART: Highly active antiretroviral therapy.

Individual: A living animal or human. In preferred embodiments, the subject is a mammal, including humans and non-human mammals such as dogs, cats, pigs, cows, sheep, goats, horses, rats, and mice. In the most preferred embodiment, the subject is a human.

Isolated: is used to describe e.g. the various GHS-R1A ligands disclosed herein, that have been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the GHS-R1A ligand will be purified.

Ligand: A molecule that binds specifically to a receptor, such as e.g. a GHS-R1A ligand, which binds specifically to the GHS-receptor 1A. Preferably, “binding specifically” between the ligand and its receptor is defined by a dissociation constant (Kd) of less than 500 nM, such as less than 100 nM, for example less than 80 nM, such as less than 60 nM, for example less than 40 nM, such as less than 20 nM, for example less than 10 nM, such as less than 5 nM, for example less than 1 nM, such as less than 0.5 nM, for example less than 0.1 nM, such as less than 0.05 nM, for example less than 0.01 nM.

Modified amino acid: an amino acid wherein an arbitrary group thereof is chemically modified. In particular, a modified amino acid chemically modified at the alpha-carbon atom in an alpha-amino acid is preferable.

Organ transplantation patient: An individual which will undergo, is undergoing or has undergone an organ transplantation, such as a transplantation of the lung, liver, kidney or heart. Accordingly, the term includes patients that will undergo organ transplantation, but are e.g. preparing for the transplantation or on a waiting list.

Palliative treatment: a treatment which relieves or soothes the symptoms of a disease or disorder but without curing the underlying disease.

Polypeptide: The phrase polypeptide refers to a molecule comprising amino acid residues which do not contain linkages other than amide linkages between adjacent amino acid residues.

Receptor: A receptor is a molecule, such as a protein, glycoprotein and the like, that can specifically (non-randomly) bind to another molecule.

REE: Resting energy expenditure. Resting energy expenditure represents the amount of energy required for a 24-hour period by the body during a non-active period.

Remission: A period during which symptoms of disease are reduced or disappear. An individual is “in remission” from a pathological condition if they are still suffering from (to any extent), or at risk of suffering from, either the symptoms or consequences of the pathological condition they suffered and/or from the effects of the treatment itself (in particular, side effects of the treatment they received). Herein, it is particularly desired that an individual “in remission” from hyperthyroidic state has a greater risk of weight gain than the average healthy individual of the same age.

Synthetic amino acid: an amino acid which is not naturally available, such as any of the synthetic amino acids shown in FIG. 1.

GH-Secretagogue: a growth hormone secretagogue, i.e. a substance stimulating growth hormone release, such as ghrelin or an analogue thereof, e.g. a GHS-R 1a ligand. Known secretagogues include L-692-429, L-692-585 (Benzoelactam compounds)

MK677 (Spiroindaner), G-7203, G-7039, G-7502 (Isonipecotic acid peptidomimetic) NN703, ipamorelin.

Secretagogue activity: the capability of a substance to stimulate growth hormone release. A suitable assay for determining secretagogue activity is described in e.g. Example 3

### Detailed Description of the GHS-R1A Ligands of the Present Invention

### “Anchor Groups”

Natural ghrelin has an anchor group comprising an acylated serine residue, which aids anchorage in the cell membrane. An anchor group is, in one embodiment of the present invention, any group capable of providing the des-acylated 28 aa human ghrelin, or an analogue thereof, with binding affinity to GHS-R 1a. In another preferred embodiment, said anchor group may be an amino acid modified with a bulky group capable of restoring secretagogue activity to des-acylated 28 aa human ghrelin. Said bulky group is preferably a hydrophobic moiety. Suitable anchor groups for use in the second aspect of the invention, in which the GHS-R 1a ligand has the general formula I′, include any bulky chemical group, preferably comprising a hydrophobic section. In the most preferred embodiment, said anchor group is capable of anchoring at least part of the structure of a GHS-R 1a ligand of the present invention in the cell membrane, most preferably in a “lipid raft” section of the membrane. In preferred embodiments, said anchor group is hydrophobic, or at least partly hydrophobic (such as an amphiphilic molecule). If an amino acid is modified to form the anchor group, any suitable amino acid may be modified with any suitable bulky group; in a preferred embodiment, a Ser residue (preferably amino acid number 3 in the amino acid chain) is modified with the bulky group. Alternatively, an amino acid group, such as in position 3 of the amino acid chain, may be replaced by a suitable anchor group. In one preferred embodiment, the anchor group is an amino acid residue modified with a fatty acid: preferred fatty acids for use in the present invention include, but are not restricted to one or more of the following: Myristic acid, Palmitic acid, Stearic acid, Oleic acid, Linoleic acid, Linonenic acid, Arachidonic acid and Eicosapentaenoic acid.

In the case when the anchor group is an amino acid that is directly modified, the amino acid thus modified preferably comprises e.g. —OH, —SH, —NH or —NH2 as a substituent group in a side chain thereof, and a group formed by acylating such a substituent group is preferred. The mode of linkage may thus be e.g. selected from the group consisting of ester, ether, thioester, thioether, amide and carbamide.

For example, if the modified amino acid is serine, threonine, tyrosine or oxyproline, the amino acid can have a hydroxyl group in the side chain. If the modified amino acid is cysteine, the amino acid can have a mercapto group in the side chain. If the modified amino acid is lysine, arginine, histidine, tryptophan, proline oroxyproline, it can have an amino group or imino group in the side chain.

The hydroxyl group, mercapto group, amino group and imino group described above can thus have been chemically modified. That is, the hydroxyl group or mercapto group can be e.g. etherized, esterified, thioetherified or thioesterified. The imino group can have e.g. been iminoetherified, iminothioetherified or alkylated. The amino group can have been e.g. amidated, thioamidated or carbamidated.

Further, the mercapto group can have been disulfidated, the imino group can have been e.g. amidated or thioamidated, and the amino group can have been e.g. alkylated or thiocarbamidated.

In a preferred embodiment the modified anchor group is Ser coupled through an ester linkage to a bulky group.

The anchor group may comprise or consist of any group with a saturated or unsaturated alkyl or acyl group containing one or more carbon atoms. In one embodiment the anchor group is an acyl group, including groups formed by removing a hydroxyl group from an organic carboxylic acid, organic sulfonic acid or organic phosphoric acid. The organic carboxylic acid includes e.g. fatty acids, and the number of carbon atoms thereof is preferably 1 to 35. In the organic sulfonic acid or organic phosphoric acid, the number of carbon atoms thereof is preferably 1 to 35.

The acyl group is preferably selected from a C1-C35 acyl group, such as a C1-C20 acyl group, such as a C1-C15 acyl group, such as a C6-C15 acyl group, such as a C6-C12 acyl group, such as a C8-C12 acyl group.

More preferably the acyl group is selected from the group of C7 acyl group, C8 acyl group, C9 acyl group, C10 acyl group, C11 acyl group, and C12 acyl group. Such acyl group may be formed from octanoic acid (preferably caprylic acid), decanoic acid (preferably capric acid), or dodecanoic acid (preferably lauric acid), as well as monoene or polyene fatty acids thereof.

In one embodiment the acyl group is selected from the group of C8 acyl group, and C10 acyl group. Such acyl groups may be formed from octanoic acid (preferably caprylic acid), or decanoic acid (preferably capric acid).

In another embodiment the acyl group is selected from the group of C7 acyl group, C9 acyl group, and C₁₁ acyl group, such as from the group of C9 acyl group and C11 acyl group.

### GHS-R1A Ligands Characterised by Novel Anchor Groups

The first aspect of the present invention relates to novel GHS-R1A ligands comprising improved anchor groups. Thus, the first aspect of the present invention relates to a GHS-R1A ligand, wherein the GHS-R1A ligand is defined by formula I

Z¹-(X¹)ₘ-(X²)-(X³)ₙ-Z², wherein
Z¹ is an optionally present protecting group;
each X¹ is independently selected from an amino acid, wherein said amino acid is selected from naturally occurring and synthetic amino acids;
and wherein X² is an anchor group, e.g. a lipid group, the anchor group preferably being either:
any amino acid selected from naturally occurring and synthetic amino acids, said amino acid being modified with a group selected from:
a) a glycerophospholipid
b) a sterol moiety
c) a sphingolipid moiety
d) a ceramide or an analogue thereof
e) an isoprenoid pyrophosphate
f) a glycosyl-phosphatidylinositol (GPI) anchor
g) a phosphatidyl serine, or analogue thereof;
   or alternatively wherein X² can preferably be selected from:
h) decenoic acid (L or D form);
i) Trp(5-NH2) (L or D form);
j) 5-hexenoic acid (L or D form)
k) 6-heptenoic acid (L or D form)
l) 7-octenoic acid (L or D form)
m) 8-nonenoic acid (L or D form)
n) Ala-3-cp (L or D form)
o) Ala-3-cb (L or D form)
p) Phe-4-Me (L or D form)
q) Phe-4-Et (L or D form)
r) Phe-4-iPr (L or D form)
s) Phe-4-Ph (L or D form)
t) Beta-MeTrp (L or D form)
u) Ala[3-(3-Quinolinyl)] (L or D form)
v) Ala[3-(2-benzimidazoyl)] (L or D form)
w) BenzoTrp (L or D form)
x) 7-AzaTrp (L or D form);
each X³ is independently selected from an amino acid, wherein said amino acid is selected from naturally occurring and synthetic amino acids,
Z² is an optionally present protecting group,
m is 0 or an integer in the range of 1-10
n is 0 or an integer in the range of 1-35;
   wherein m and n cannot both be 0.

Accordingly, the GHS-R1A ligand includes the naturally occurring 28 aa human ghrelin, the amino acid of which is shown in SEQ ID NO: 1, modified with an anchor group according to the present invention, as well as the naturally occurring 27 aa human ghrelin, the amino acid of which is shown in SEQ ID NO: 2, modified with an anchor group according to the present invention.

GHS-R 1a ligands of the invention may be in the form of diastereomers as well as their racemic and resolved enantiomerically pure forms. A GHS-R 1a ligand of the invention can contain D-amino acids, L-amino acids, alpha-amino acid, beta-amino acid, gamma-amino acid, natural amino acid and synthetic amino acid or the like or a combination thereof. Preferably, amino acids present in a GHS-R1A ligand of the invention are the L-enantiomer or the D-enantiomer.

The number of amino acids N-terminally to the X² amino acid is preferably within the range of 1-9. Accordingly, m is preferably an integer in the range of 1-9, such as of 1-8, such as of 1-7, such as of 1-6, such as of 1-5, such as of 1-4, such as of 1-3, such as of 1-2, such as 2.

It is more preferred that the number of amino acids N-terminally to the X² amino acid is low, such as of 1-3, such as of 1-2. Most preferably 2 amino acids are positioned N-terminal to the modified amino acid.

In a preferred embodiment (X¹)ₘ has a Gly residue in the N-terminal part of the sequence. Accordingly, in a preferred embodiment (X¹)ₘ is selected from the sequences:

Gly, Gly-Ser, Gly-Cys, Gly-Lys, Gly-Asp, Gly-Glu,
Gly-Arg, Gly-His, Gly-Asn, Gly-Gln, Gly-Thr,
and
Gly-Tyr.

More preferably (X¹)ₘ is selected from Gly-Ser, and Gly-Cys, most preferably from Gly-Ser.

It is preferred that X² is selected from the group of modified Ser, modified Cys and modified Lys, such as wherein X² is modified Ser.

In other words, in a preferred embodiment the GHS-R1A ligand is selected from a compound of

Z¹-Gly-(X¹)ₘ₋₁-(X²)-(X³)ₙ-Z², formula II

Z¹-Gly-Ser-(X²)-(X³)ₙ-Z², and formula III

Z¹-Gly-(X²)-(X³)ₙ-Z². formula IV

And more preferably the GHS-R1A ligand has formula III.

### Preferred Embodiments of X²

In a preferred embodiment X² is an anchor group being either:
any amino acid selected from naturally occurring and synthetic amino acids, said amino acid being modified with a group selected from:
a) a glycerophospholipid
b) a sterol moiety
c) a sphingolipid moiety
d) a ceramide or an analogue thereof
e) an isoprenoid pyrophosphate
f) a glycosyl-phosphatidylinositol (GPI) anchor
g) a phosphatidyl serine, or analogue thereof;
   or alternatively wherein X² is selected from:
h) decenoic acid (L or D form);
i) Trp(5-NH2) (L or D form); (one preferred structure shown below)

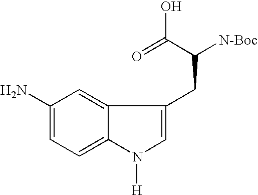

j) 5-hexenoic acid (L or D form) (one preferred structure shown below)

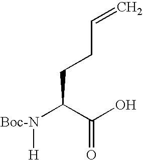

k) 6-heptenoic acid (L or D form) (one preferred structure shown below)

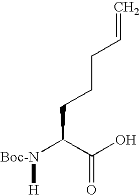

l) 7-octenoic acid (L or D form) (one preferred structure shown below)

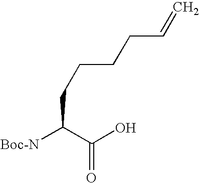

m) 8-nonenoic acid (L or D form) (one preferred structure shown below)

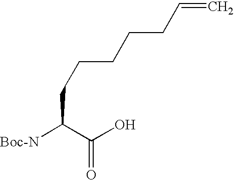

n) Ala-3-cp (L or D form) (one preferred structure shown below)

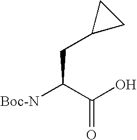

o) Ala-3-cb (L or D form) (one preferred structure shown below)

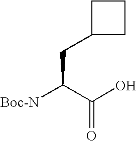

p) Phe-4-Me (L or D form) (one preferred structure shown below)

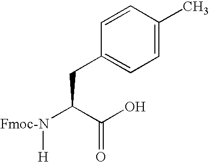

q) Phe-4-Et (L or D form) (one preferred structure shown below)

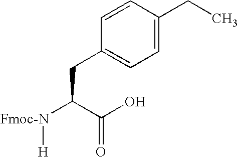

r) Phe-4-iPr (L or D form) (one preferred structure shown below)

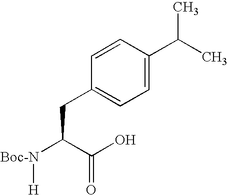

s) Phe-4-Ph (L or D form) (one preferred structure shown below)

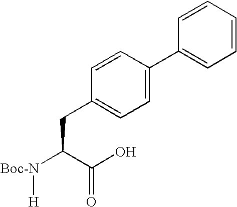

t) Beta-MeTrp (L or D form) (one preferred structure shown below)

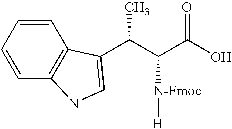

u) Ala[3-(3-Quinolinyl)] (L or D form) (one preferred structure shown below)

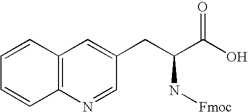

v) Ala[3-(2-benzimidazoyl)] (L or D form) (one preferred structure shown below)

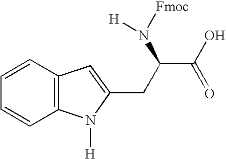

w) BenzoTrp (L or D form) (one preferred structure shown below)

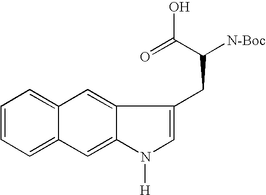

x) 7-AzaTrp (L or D form) (one preferred structure shown below);

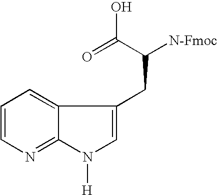

In one preferred embodiment, X² is any amino acid selected from naturally occurring and synthetic amino acids, said amino acid being modified with a group selected from:
a) a glycerophospholipid
b) a sterol moiety
c) a sphingolipid moiety
d) a ceramide or an analogue thereof
e) an isoprenoid pyrophosphate
f) a glycosyl-phosphatidylinositol (GPI) anchor
g) a phosphatidyl serine, or analogue thereof;
   such as selected from one of the following groups:
   a)+f)+g);
   b)+c)+d);
   a)+f);
   e)+f);
   a)+g)

In another preferred embodiment, X² is selected from:
h) decenoic acid (L or D form)
i) Trp(5-NH2) (L or D form);
j) 5-hexenoic acid (L or D form)
k) 6-heptenoic acid (L or D form)
l) 7-octenoic acid (L or D form)
m) 8-nonenoic acid (L or D form)
n) Ala-3-cp (L or D form)
o) Ala-3-cb (L or D form)
p) Phe-4-Me (L or D form)
q) Phe-4-Et (L or D form)
r) Phe-4-iPr (L or D form)
s) Phe-4-Ph (L or D form)
t) Beta-MeTrp (L or D form)
u) Ala[3-(3-Quinolinyl)] (L or D form)
v) Ala[3-(2-benzimidazoyl)] (L or D form)
w) BenzoTrp (L or D form)
x) 7-AzaTrp (L or D form)
   such as selected from one of the following groups:
   i)+j)+k)+l)+m); n)+o)+p)+q)+r)+s)+t)+u)+v)+w)+x);
   h)+j)+k)+l)+m);
   j)+k)+l)+m);
   p)+q)+r)+s);
   n)+o)+u)+v);
   t)+w)+x).

In one preferred embodiment, the X² group is decenoic acid, or a variant thereof. More preferably, the compound of the present invention has a sequence according to SEQ ID NO: 6.

In another preferred embodiment, the anchor group is an amino acid modified with a glycerophospholipid, and preferably has the basic structure shown below, wherein R1 and R2 represent saturated and/or unsaturated hydrocarbon chains (preferably C1-C30), and X is the amino acid moiety.

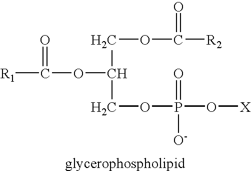

In another preferred embodiment, the anchor group is an amino acid modified with a sterol moiety, said sterol being preferably selected from one or more of the following: Cholesterol, Stigmasterol, Ergosterol, Androstenol and Lanosterol, more preferably cholesterol. In a specific embodiment the GHS-R 1a ligand according to the present invention has the sequence according to SEQ ID NO: 13.

In another preferred embodiment, the anchor group is an amino acid modified with a sphingolipid moiety. Preferably, said sphingolipid comprises a derivative of the lipid sphingosine or dihydrosphingosine, e.g. with one of the structures given below:

### Sphingosine: CH₃—(CH₂)₁₂—CH═CH—HCOH—HCNH₂—CH₂—OH

### Dihydrosphingosine: CH₃—(CH₂)₁₄—HCOH—HCNH₂—CH₂—OH

In another preferred embodiment, the anchor group is an amino acid modified with ceramide or an analogue thereof. Preferably, said ceramide analog of ceramide has the basic structure shown below, wherein R represents one or more saturated and/or unsaturated hydrocarbon chains (preferably C1-C30).

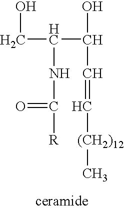

In another preferred embodiment, the anchor group is an amino acid modified with an isoprenoid. One preferred isoprenoid is a farnesyl residue, such as shown in the structure below:

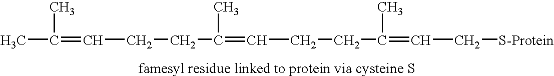

In another preferred embodiment, the anchor group is an amino acid modified with an isoprenoid pyrophosphate. Preferably, said isoprenoid pyrophosphate is farnesyl pyrophosphate, with structure shown below.

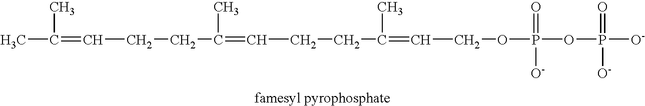

In another preferred embodiment, the anchor group is an amino acid modified with a Glycosyl-phosphatidylinositol (GPI) anchor. It is contemplated that such anchor groups provide improved anchorage in the cellular membrane over various known GH secretagogues. Preferably, said GPI anchor comprises a phosphatidyl inositol moiety linked through glucosamine and mannose to a phosphoryl ethanolamine residue that is linked to the C terminal amino acid of the protein by its amino group.

In another preferred embodiment, the anchor group can consist of more than one anchor portion, such as for example in the case of phosphatidyl serine, which consists of two anchor portions. Thus, in one preferred embodiment, the anchor group is an amino acid modified with phosphatidyl serine, or analogue thereof. More preferably, the GHS-R1A ligand of the present invention has sequence according to SEQ ID NO: 5.

It is preferred that X² is selected from the group of modified Ser, modified Cys and modified Lys, such as wherein X² is modified Ser.

In particular X² is selected from the group of modified Ser, Cys, Asp, Lys, Trp, Phe, Ile, and Leu. More preferably X² is selected from the group of modified Ser, modified Cys and modified Lys, and most preferably X² is modified Ser.

Furthermore, (X¹)ₘ-(X²) is preferably Gly-Xaa-Ser*, or Gly-Xaa-Cys*, wherein Xaa is any amino acid, more preferably (X¹)ₘ-(X²) is Gly-Ser-Ser*, or Gly-Ser-Cys*, wherein * indicates that the amino acid residue is modified to form the anchor group.

In one embodiment of the present invention, the anchor is built directly into the peptide chain (as in the case, e.g. when phosphatidyl serine constitutes the anchor group). In another embodiment, the GHS′R 1a ligand is manufactured by joining the anchor group to an amino acid residue of the ligand by acylation or another suitable chemical reaction for coupling the anchor group in question to the amino acid backbone of the ligand.

### Preferred Embodiments of (X³)ₙ

(X³)ₙ preferably comprises a sequences which is a fragment of ghrelin, such as human ghrelin.

In one embodiment the length of the GHS-R1A ligand is substantially similar to the length of human ghrelin, i.e. 27 or 28 amino acids. Accordingly, n is preferably an integer in the range of 1-25, such as 1-24, such as 1-15, such as 1-10, or such as 10-25, such as 10-24, such as 15-25, such as 15-24.

Most preferably a GHS-R1A ligand to be modified to incorporate an anchor group in accordance with the present invention includes the naturally occurring 28 aa human ghrelin, the amino acid of which is shown in SEQ ID NO: 1, as well as the naturally occurring 27 aa human ghrelin, the amino acid of which is shown in SEQ ID NO: 2.

In another embodiment (X³)ₙ may be selected from any fragment of ghrelin, such as human ghrelin, and accordingly, (X³)ₙ may be selected from one or more of the sequences shown below or a homologue thereof:

(SEQ ID NO: 35)

Phe Leu Ser Pro Glu His Gln Arg Val Gln Gln Arg
Lys Glu Ser Lys Lys Pro Pro Ala Lys Leu Gln Pro
Arg

(SEQ ID NO: 36)

Phe Leu Ser Pro Glu His Gln Arg Val Gln Gln Arg
Lys Glu Ser Lys Lys Pro Pro Ala Lys Leu Gln Pro

(SEQ ID NO: 37)

Phe Leu Ser Pro Glu His Gln Arg Val Gln Gln Arg
Lys Glu Ser Lys Lys Pro Pro Ala Lys Leu Gln

(SEQ ID NO: 38)

Phe Leu Ser Pro Glu His Gln Arg Val Gln Gln Arg
Lys Glu Ser Lys Lys Pro Pro Ala Lys Leu

(SEQ ID NO: 39)

Phe Leu Ser Pro Glu His Gln Arg Val Gln Gln Arg
Lys Glu Ser Lys Lys Pro Pro Ala Lys

(SEQ ID NO: 40)

Phe Leu Ser Pro Glu His Gln Arg Val Gln Gln Arg
Lys Glu Ser Lys Lys Pro Pro Ala

(SEQ ID NO: 41)

Phe Leu Ser Pro Glu His Gln Arg Val Gln Gln Arg
Lys Glu Ser Lys Lys Pro Pro

(SEQ ID NO: 42)

Phe Leu Ser Pro Glu His Gln Arg Val Gln Gln Arg
Lys Glu Ser Lys Lys Pro

(SEQ ID NO: 43)

Phe Leu Ser Pro Glu His Gln Arg Val Gln Gln Arg
Lys Glu Ser Lys Lys

(SEQ ID NO: 44)

Phe Leu Ser Pro Glu His Gln Arg Val Gln Gln Arg
Lys Glu Ser Lys

(SEQ ID NO: 45)

Phe Leu Ser Pro Glu His Gln Arg Val Gln Gln Arg
Lys Glu Ser

(SEQ ID NO: 46)

Phe Leu Ser Pro Glu His Gln Arg Val Gln Gln Arg
Lys Glu

(SEQ ID NO: 47)

Phe Leu Ser Pro Glu His Gln Arg Val Gln Gln Arg
Lys

(SEQ ID NO: 48)

Phe Leu Ser Pro Glu His Gln Arg Val Gln Gln Arg

(SEQ ID NO: 49)

Phe Leu Ser Pro Glu His Gln Arg Val Gln Gln

(SEQ ID NO: 50)

Phe Leu Ser Pro Glu His Gln Arg Val Gln

(SEQ ID NO: 51)

Phe Leu Ser Pro Glu His Gln Arg Val

(SEQ ID NO: 52)

Phe Leu Ser Pro Glu His Gln Arg

(SEQ ID NO: 30)

Phe Leu Ser Pro Glu His Gln

(SEQ ID NO: 31)

Phe Leu Ser Pro Glu His

(SEQ ID NO: 32)

Phe Leu Ser Pro Glu

(SEQ ID NO: 33)

Phe Leu Ser Pro

(SEQ ID NO: 34)

Phe Leu Ser
Phe Leu
Phe

Or selected from

(SEQ ID NO: 82)

Phe Leu Ser Pro Glu His Gln Lys Val Gln Gln Arg
Lys Glu Ser Lys Lys Pro Pro Ala Lys Leu Gln Pro
Arg

(SEQ ID NO: 83)

Phe Leu Ser Pro Glu His Gln Lys Val Gln Gln Arg
Lys Glu Ser Lys Lys Pro Pro Ala Lys Leu Gln Pro

(SEQ ID NO: 84)

Phe Leu Ser Pro Glu His Gln Lys Val Gln Gln Arg
Lys Glu Ser Lys Lys Pro Pro Ala Lys Leu Gln

(SEQ ID NO: 85)

Phe Leu Ser Pro Glu His Gln Lys Val Gln Gln Arg
Lys Glu Ser Lys Lys Pro Pro Ala Lys Leu

(SEQ ID NO: 86)

Phe Leu Ser Pro Glu His Gln Lys Val Gln Gln Arg
Lys Glu Ser Lys Lys Pro Pro Ala Lys

(SEQ ID NO: 87)

Phe Leu Ser Pro Glu His Gln Lys Val Gln Gln Arg
Lys Glu Ser Lys Lys Pro Pro Ala

(SEQ ID NO: 88)

Phe Leu Ser Pro Glu His Gln Lys Val Gln Gln Arg
Lys Glu Ser Lys Lys Pro Pro

(SEQ ID NO: 89)

Phe Leu Ser Pro Glu His Gln Lys Val Gln Gln Arg
Lys Glu Ser Lys Lys Pro

(SEQ ID NO: 90)

Phe Leu Ser Pro Glu His Gln Lys Val Gln Gln Arg
Lys Glu Ser Lys Lys

(SEQ ID NO: 91)

Phe Leu Ser Pro Glu His Gln Lys Val Gln Gln Arg
Lys Glu Ser Lys

(SEQ ID NO: 92)

Phe Leu Ser Pro Glu His Gln Lys Val Gln Gln Arg
Lys Glu Ser

(SEQ ID NO: 93)

Phe Leu Ser Pro Glu His Gln Lys Val Gln Gln Arg
Lys Glu

(SEQ ID NO: 94)

Phe Leu Ser Pro Glu His Gln Lys Val Gln Gln Arg
Lys

(SEQ ID NO: 95)

Phe Leu Ser Pro Glu His Gln Lys Val Gln Gln Arg

(SEQ ID NO: 96)

Phe Leu Ser Pro Glu His Gln Lys Val Gln Gln

(SEQ ID NO: 97)

Phe Leu Ser Pro Glu His Gln Lys Val Gln

(SEQ ID NO: 98)

Phe Leu Ser Pro Glu His Gln Lys Val

(SEQ ID NO: 99)

Phe Leu Ser Pro Glu His Gln Lys

Or selected from

(SEQ ID NO: 100)

Phe Leu Ser Pro Glu His Gln Arg Ala Gln Gln Arg
Lys Glu Ser Lys Lys Pro Pro Ala Lys Leu Gln Pro
Arg

(SEQ ID NO: 101)

Phe Leu Ser Pro Glu His Gln Arg Ala Gln Gln Arg
Lys Glu Ser Lys Lys Pro Pro Ala Lys Leu Gln Pro

(SEQ ID NO: 102)

Phe Leu Ser Pro Glu His Gln Arg Ala Gln Gln Arg
Lys Glu Ser Lys Lys Pro Pro Ala Lys Leu Gln

(SEQ ID NO: 103)

Phe Leu Ser Pro Glu His Gln Arg Ala Gln Gln Arg
Lys Glu Ser Lys Lys Pro Pro Ala Lys Leu

(SEQ ID NO: 104)

Phe Leu Ser Pro Glu His Gln Arg Ala Gln Gln Arg
Lys Glu Ser Lys Lys Pro Pro Ala Lys

(SEQ ID NO: 105)

Phe Leu Ser Pro Glu His Gln Arg Ala Gln Gln Arg
Lys Glu Ser Lys Lys Pro Pro Ala

(SEQ ID NO: 106)

Phe Leu Ser Pro Glu His Gln Arg Ala Gln Gln Arg
Lys Glu Ser Lys Lys Pro Pro

(SEQ ID NO: 107)

Phe Leu Ser Pro Glu His Gln Arg Ala Gln Gln Arg
Lys Glu Ser Lys Lys Pro

(SEQ ID NO: 108)

Phe Leu Ser Pro Glu His Gln Arg Ala Gln Gln Arg
Lys Glu Ser Lys Lys

(SEQ ID NO: 109)

Phe Leu Ser Pro Glu His Gln Arg Ala Gln Gln Arg
Lys Glu Ser Lys

(SEQ ID NO: 110)

Phe Leu Ser Pro Glu His Gln Arg Ala Gln Gln Arg
Lys Glu Ser

(SEQ ID NO: 111)

Phe Leu Ser Pro Glu His Gln Arg Ala Gln Gln Arg
Lys Glu

(SEQ ID NO: 112)

Phe Leu Ser Pro Glu His Gln Arg Ala Gln Gln Arg
Lys

(SEQ ID NO: 113)

Phe Leu Ser Pro Glu His Gln Arg Ala Gln Gln Arg

(SEQ ID NO: 114)

Phe Leu Ser Pro Glu His Gln Arg Ala Gln Gln

(SEQ ID NO: 115)

Phe Leu Ser Pro Glu His Gln Arg Ala Gln

(SEQ ID NO: 116)

Phe Leu Ser Pro Glu His Gln Arg Ala

Or selected from

(SEQ ID NO: 117)

Phe Leu Ser Pro Glu His Gln Lys Ala Gln Gln Arg
Lys Glu Ser Lys Lys Pro Pro Ala Lys Leu Gln Pro
Arg

(SEQ ID NO: 118)

Phe Leu Ser Pro Glu His Gln Lys Ala Gln Gln Arg
Lys Glu Ser Lys Lys Pro Pro Ala Lys Leu Gln Pro

(SEQ ID NO: 119)

Phe Leu Ser Pro Glu His Gln Lys Ala Gln Gln Arg
Lys Glu Ser Lys Lys Pro Pro Ala Lys Leu Gln

(SEQ ID NO: 120)

Phe Leu Ser Pro Glu His Gln Lys Ala Gln Gln Arg
Lys Glu Ser Lys Lys Pro Pro Ala Lys Leu

(SEQ ID NO: 121)

Phe Leu Ser Pro Glu His Gln Lys Ala Gln Gln Arg
Lys Glu Ser Lys Lys Pro Pro Ala Lys

(SEQ ID NO: 122)

Phe Leu Ser Pro Glu His Gln Lys Ala Gln Gln Arg
Lys Glu Ser Lys Lys Pro Pro Ala

(SEQ ID NO: 123)

Phe Leu Ser Pro Glu His Gln Lys Ala Gln Gln Arg
Lys Glu Ser Lys Lys Pro Pro

(SEQ ID NO: 123)

Phe Leu Ser Pro Glu His Gln Lys Ala Gln Gln Arg
Lys Glu Ser Lys Lys Pro

(SEQ ID NO: 124)

Phe Leu Ser Pro Glu His Gln Lys Ala Gln Gln Arg
Lys Glu Ser Lys Lys

(SEQ ID NO: 125)

Phe Leu Ser Pro Glu His Gln Lys Ala Gln Gln Arg
Lys Glu Ser Lys

(SEQ ID NO: 127)

Phe Leu Ser Pro Glu His Gln Lys Ala Gln Gln Arg
Lys Glu Ser

(SEQ ID NO: 128)

Phe Leu Ser Pro Glu His Gln Lys Ala Gln Gln Arg
Lys Glu

(SEQ ID NO: 129)

Phe Leu Ser Pro Glu His Gln Lys Ala Gln Gln Arg
Lys

(SEQ ID NO: 130)

Phe Leu Ser Pro Glu His Gln Lys Ala Gln Gln Arg

(SEQ ID NO: 131)

Phe Leu Ser Pro Glu His Gln Lys Ala Gln Gln

(SEQ ID NO: 132)

Phe Leu Ser Pro Glu His Gln Lys Ala Gln

(SEQ ID NO: 52)

Phe Leu Ser Pro Glu His Gln Lys Ala

In another embodiment (X³)ₙ comprises or consists of a sequence selected from the sequences

| | |
|---|---|
| Phe Leu Ser Pro Glu His Gln | (SEQ ID NO: 30) |
| Phe Leu Ser Pro Glu His | (SEQ ID NO: 31) |
| Phe Leu Ser Pro Glu | (SEQ ID NO: 32) |
| Phe Leu Ser Pro | (SEQ ID NO: 33) |
| Phe Leu Ser | (SEQ ID NO: 34) |
| Phe Leu | |
| Phe | |

### Novel GHS-R1A Ligands Comprising at Least One D-Amino Acid

In a second aspect of the present invention, a GHS-R1A ligand or a pharmaceutically acceptable salt thereof is provided,
wherein the GHS-R1A ligand has a structure defined by formula I′

Z²-(X³)ₙ-(X²)-(X¹)ₘ-Z³-Z¹ formula I′
wherein Z¹ is an optionally present protecting group,
each X¹ is an amino acid independently selected from naturally occurring and synthetic amino acids,
X² is an anchor group, preferably selected from naturally occurring and synthetic amino acids modified with a bulky group, more preferably a modified D-amino acid,
each X³ is independently selected from an amino acid, wherein said amino acid is selected from naturally occurring and synthetic amino acids, with the proviso that at least one (X³) is a D-amino acid,
Z² is an optionally present protecting group,
Z³ is an optionally present linker or C-terminal group,
m is 0 or an integer in the range of 1-3
n is 0 or an integer in the range of 1-35,
and wherein both n and m cannot be 0.

It is preferred that Z² is selected from the group consisting of:
a) βAla or
b) X¹-βAla or
c) GABA or
d) X1-GABA or
e) -X1-Aminopentanoyl or
f) hydroxy acetic acid (HAA) or
g) X1-HAA or
h) a compound with formula B, shown below:

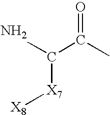

i) a compound with the formula i) shown below:
j) a compound with the formula j) shown below:

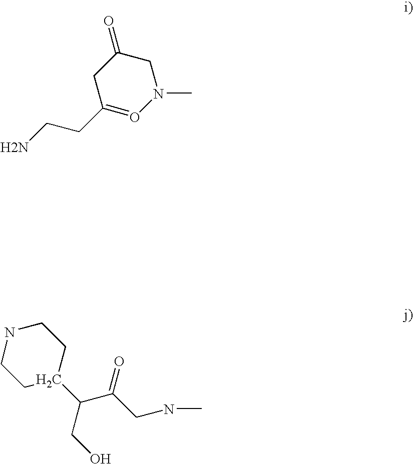

wherein X7 is a spacer with a length of 1-8 chemical bonds, and X8 is a hydrogen bond donor, such as an amine or hydroxyl group; wherein m is 2. More preferably, Z² is selected from compounds b)-h).

In preferred embodiments, X² is selected from the group of modified Ser, modified Cys and modified Lys, such as wherein X² is modified Ser.

The number of amino acids N-terminally to the X² amino acid is preferably within the range of 1-9. Accordingly, m is preferably an integer in the range of 1-9, such as of from 1-8, such as of from 1-7, such as of from 1-6, such as of from 1-5, such as of from 1-4, such as of from 1-3, such as of from 1-2, such as 2.

It is more preferred that the number of amino acids N-terminally to the X² amino acid is low, such as of from 1-3, such as of from 1-2. Most preferably 2 amino acids are positioned N-terminal to the modified amino acid.

In a preferred embodiment (X¹)ₘ has a Gly residue in the N-terminal part of the sequence. Accordingly, in preferred embodiment (X¹)ₘ is selected from the sequences:

Gly, Gly-Ser, Gly-Cys, Gly-Lys, Gly-Asp, Gly-Glu,
Gly-Arg, Gly-His, Gly-Asn, Gly-Gln, Gly-Thr,
and
Gly-Tyr.

More preferably (X¹)ₘ is selected from Gly-Ser, and Gly-Cys, most preferably from Gly-Ser.

It is preferred that X² is selected from the group of modified Ser, modified Cys and modified Lys, such as wherein X² is modified Ser.

For example, said compound has one of the following sequence identifications numbers: SEQ ID NO: 20-29, or is an analogue, homologue or variant thereof.

In further preferred embodiments, the compound is selected from a compound of:

Z²-(X³)ₙ-(X²)-(X¹)ₘ₋₁-Gly-Z¹, and formula II′

Z²-(X³)ₙ-(X²)-D-Ser-Gly-Z¹, and formula III′

Z²-(X³)ₙ-(X²)-Gly-Z¹ formula IV′

Z²-(X³)ₙ-D-Ser-Z³-Z¹ formula V′

More preferably, the GHS-R1A ligand is having formula III.

In preferred embodiments, a GHS-R 1a ligand of the invention comprises a structure having the formula VI′:

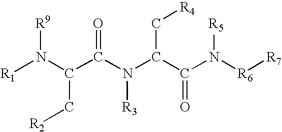

wherein:
R₁ is an alcohol, ether, hydrocarbon, hydrazine, peptide or peptidomimetic moiety,
R₂ is an aromatic moiety,

### R₃ is H or CH₃,

R₄ is an aromatic, hydrophobic or amphiphilic moiety, such as an anchor group as described herein,

### R₅ is H or CH₃,

R₆ is a spacer with length of 1-8 chemical bonds, preferably 4-5 chemical bonds,
R₇ is a hydrogen bond donor, such as NH or SH or (more preferably) NH₂ or OH,
R⁹ is preferably H.

Preferred alcohols include, but are not restricted to, ethanol, glycerol and phenols. Preferred ethers include, but are not restricted to dimethylether and methyl ethyl ether. Preferred hydrocarbons include, but are not restricted to, saturated hydrocarbons with the length of 1-4 chemical bonds. Preferred hydrazines include, but are not restricted to, hydrazine, 1,1-dimethylhydrazine, and 1,2-dimethylhydrazine. Preferred peptides include, but are not restricted to, a small peptide with the length of 1 or 2 amino acid. Preferred peptidomimetic moieties include, but are not restricted to, a small mimetic with the length of 1 or 2 amino acids, such as a peptoid or reduced peptide bond.

In one preferred embodiment, (X³)ₙ comprises a sequence selected from one or more of the sequences shown below:

(SEQ ID NO: 53)

D-Gln D-His D-Glu D-Pro D-Ser D-Leu D-Phe

(SEQ ID NO: 54)

D-His D-Glu D-Pro D-Ser D-Leu D-Phe

(SEQ ID NO: 55)

D-Glu D-Pro D-Ser D-Leu D-Phe

(SEQ ID NO: 56)

D-Pro D-Ser D-Leu D-Phe

(SEQ ID NO: 57)

D-Ser D-Leu D-Phe
D-Leu D-Phe
D-Phe

n is preferably an integer in the range of 1-25, such as 1-24, such as 1-15, such as 1-10, such as 10-25, such as 10-24, such as 15-25, such as 15-24.

In other preferred embodiments, (X3)n is selected from one or more of the sequences shown below:

(SEQ ID NO: 58)

D-Arg D-Pro D-Gln D-Leu D-Lys D-Ala D-Pro D-Pro
D-Lys D-Lys D-Ser D-Glu D-Lys D-Arg D-Gln D-Gln
D-Val D-Arg D-Gln D-His D-Glu D-Pro D-Ser D-Leu
D-Phe

(SEQ ID NO: 59)

D-Pro D-Gln D-Leu D-Lys D-Ala D-Pro D-Pro D-Lys
D-Lys D-Ser D-Glu D-Lys D-Arg D-Gln D-Gln D-Val
D-Arg D-Gln D-His D-Glu D-Pro D-Ser D-Leu D-Phe

(SEQ ID NO: 60)

D-Gln D-Leu D-Lys D-Ala D-Pro D-Pro D-Lys D-Lys
D-Ser D-Glu D-Lys D-Arg D-Gln D-Gln D-Val D-Arg
D-Gln D-His D-Glu D-Pro D-Ser D-Leu D-Phe

(SEQ ID NO: 61)

D-Leu D-Lys D-Ala D-Pro D-Pro D-Lys D-Lys D-Ser
D-Glu D-Lys D-Arg D-Gln D-Gln D-Val D-Arg D-Gln
D-His D-Glu D-Pro D-Ser D-Leu D-Phe

(SEQ ID NO: 62)

D-Lys D-Ala D-Pro D-Pro D-Lys D-Lys D-Ser D-Glu
D-Lys D-Arg D-Gln D-Gln D-Val D-Arg D-Gln D-His
D-Glu D-Pro D-Ser D-Leu D-Phe

(SEQ ID NO: 63)

D-Arg D-Pro D-Gln D-Leu D-Lys D-Ala D-Pro D-Pro
D-Lys D-Lys D-Ser D-Glu D-Lys D-Arg D-Gln D-Gln
D-Val D-Arg D-Gln D-His D-Glu D-Pro D-Ser D-Leu
D-Phe

(SEQ ID NO: 64)

D-Pro D-Gln D-Leu D-Lys D-Ala D-Pro D-Pro D-Lys
D-Lys D-Ser D-Glu D-Lys D-Arg D-Gln D-Gln D-Val
D-Arg D-Gln D-His D-Glu D-Pro D-Ser D-Leu D-Phe

(SEQ ID NO: 65)

D-Gln D-Leu D-Lys D-Ala D-Pro D-Pro D-Lys D-Lys
D-Ser D-Glu D-Lys D-Arg D-Gln D-Gln D-Val D-Arg
D-Gln D-His D-Glu D-Pro D-Ser D-Leu D-Phe

(SEQ ID NO: 66)

D-Leu D-Lys D-Ala D-Pro D-Pro D-Lys D-Lys D-Ser
D-Glu D-Lys D-Arg D-Gln D-Gln D-Val D-Arg D-Gln
D-His D-Glu D-Pro D-Ser D-Leu D-Phe

(SEQ ID NO: 67)

D-Lys D-Ala D-Pro D-Pro D-Lys D-Lys D-Ser D-Glu
D-Lys D-Arg D-Gln D-Gln D-Val D-Arg D-Gln D-His
D-Glu D-Pro D-Ser D-Leu D-Phe

(SEQ ID NO: 68)

D-Ala D-Pro D-Pro D-Lys D-Lys D-Ser D-Glu D-Lys
D-Arg D-Gln D-Gln D-Val D-Arg D-Gln D-His D-Glu
D-Pro D-Ser D-Leu D-Phe

(SEQ ID NO: 69)

D-Pro D-Pro D-Lys D-Lys D-Ser D-Glu D-Lys D-Arg
D-Gln D-Gln D-Val D-Arg D-Gln D-His D-Glu D-Pro
D-Ser D-Leu D-Phe

(SEQ ID NO: 70)

D-Pro D-Lys D-Lys D-Ser D-Glu D-Lys D-Arg D-Gln
D-Gln D-Val D-Arg D-Gln D-His D-Glu D-Pro D-Ser
D-Leu D-Phe

(SEQ ID NO: 71)

D-Lys D-Lys D-Ser D-Glu D-Lys D-Arg D-Gln D-Gln
D-Val D-Arg D-Gln D-His D-Glu D-Pro D-Ser D-Leu
D-Phe

(SEQ ID NO: 72)

D-Lys D-Ser D-Glu D-Lys D-Arg D-Gln D-Gln D-Val
D-Arg D-Gln D-His D-Glu D-Pro D-Ser D-Leu D-Phe

(SEQ ID NO: 73)

D-Ser D-Glu D-Lys D-Arg D-Gln D-Gln D-Val D-Arg
D-Gln D-His D-Glu D-Pro D-Ser D-Leu D-Phe

(SEQ ID NO: 74)

D-Glu D-Lys D-Arg D-Gln D-Gln D-Val D-Arg D-Gln
D-His D-Glu D-Pro D-Ser D-Leu D-Phe

(SEQ ID NO: 75)

D-Lys D-Arg D-Gln D-Gln D-Val D-Arg D-Gln D-His
D-Glu D-Pro D-Ser D-Leu D-Phe

(SEQ ID NO: 76)

D-Arg D-Gln D-Gln D-Val D-Arg D-Gln D-His D-Glu
D-Pro D-Ser D-Leu D-Phe

(SEQ ID NO: 77)

D-Gln D-Gln D-Val D-Arg D-Gln D-His D-Glu D-Pro
D-Ser D-Leu D-Phe

(SEQ ID NO: 78)

D-Gln D-Val D-Arg D-Gln D-His D-Glu D-Pro D-Ser
D-Leu D-Phe

(SEQ ID NO: 79)

D-Val D-Arg D-Gln D-His D-Glu D-Pro D-Ser D-Leu
D-Phe

(SEQ ID NO: 80)

D-Arg D-Gln D-His D-Glu D-Pro D-Ser D-Leu D-Phe

(SEQ ID NO: 53)

D-Gln D-His D-Glu D-Pro D-Ser D-Leu D-Phe

(SEQ ID NO: 54)

D-His D-Glu D-Pro D-Ser D-Leu D-Phe

(SEQ ID NO: 55)

D-Glu D-Pro D-Ser D-Leu D-Phe

(SEQ ID NO: 56)

D-Pro D-Ser D-Leu D-Phe

(SEQ ID NO: 57)

D-Ser D-Leu D-Phe
D-Leu D-Phe
D-Phe

The anchor group suitable for this second aspect of the present invention is, in one embodiment of the present invention, any group described in the sections entitled “anchor groups”, above. Further preferred anchor groups for use in the aspect described herein include any of the anchor groups mentioned in the section above with the heading “Preferred embodiments of X²”

In a preferred embodiment, a GHS-R1A ligand of the present invention has the sequence according to SEQ ID NO: 4. In another preferred embodiment, a GHS-R1A ligand of the present invention has the sequence according to SEQ ID NO: 10.

### GHS-R1A Ligands Comprising Motif A

In the third aspect of the present invention, a GHS-R1A ligand, or a pharmaceutically acceptable salt thereof, is provided,
wherein the GHS-R1A ligand has a structure defined by formula I″:

Z¹-X¹-X²-X³-X⁴-X⁵-X⁶-Z², Formula I″

wherein
Z¹ is an optionally present protecting group;
X¹ is an amino acid having a structure defined by motif A:

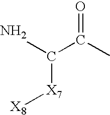

X², X³ and X⁵ are aromatic amino acids independently selected from naturally occurring and synthetic amino acids,
X⁴ is an optionally present amino acid selected from naturally occurring and synthetic amino acids,
X⁶ is optionally present and selected from the group consisting of:
a) an alcohol
b) an ether
c) a hydrocarbon
d) a hydrazine
e) a peptide
f) a peptidomimetic moiety;
X₇ is a spacer with length of 1-8 chemical bonds, such as with the length of 3-6 bonds, or preferably 4-5 chemical bonds;
X₈ is a hydrogen bond donor;
and Z² is an optionally present protecting group,
   with the proviso that at least one of X¹-X⁵ is a D-amino acid, such as at least two are D-amino acids. In one preferred embodiment, two of X¹-X⁵ are D-amino acids. In another preferred embodiment, three of X¹-X⁵ are D-amino acids. In another preferred embodiment, four of X¹-X⁵ are D-amino acids. In another preferred embodiment, five of X¹-X⁵ are D-amino acids.

In preferred embodiments, X¹ is lysine, such as D-lysine.

It is furthermore preferred that X², X³ and X⁵ are independently selected from the group consisting of the following:
(a) phenylalanine
(b) tryptophan
(c) tyrosine
(d) napthylalanine
(e) methyltryptophan
(f) aminoisobutyric acid;
   such as being selected from one of the following groups:
   (a)+(b);
   (b)+(e);
   (c)+(f);
   (d)+(e).

In another preferred embodiment, X² is phenylalanine, such as L-phenylalanine.

In another preferred embodiment, X² is napthylalanine.

In another preferred embodiment, X³ is Tryptophan, such as D-Tryptophan.

In another preferred embodiment, X³ is napthylalanine.

In another preferred embodiment, X⁵ is napthylalanine.

In another preferred embodiment, X⁵ is tryptophan, such as L-tryptophan.

In another preferred embodiment, X⁵ is 2-methyltryptophan.

In another preferred embodiment, X⁵ is aminoisobutyric acid.

In another preferred embodiment, X⁶ is alanine, such as D-alanine.

In another preferred embodiment, X⁶ is Histidine, such as D-histidine.

In another preferred embodiment, X⁶ is His-Ala, such as D-His-D-Ala.

In another preferred embodiment, X⁴ is a hydrophilic amino acid. Preferably, said hydrophilic amino acid is selected from the group consisting of:
(a) arginine
(b) asparagine
(c) aspartic acid
(d) glutamic acid
(e) lysine
(f) threonine
(g) serine
(h) glutamine;
such as selected from one of the groups consisting of:
(a)+(b)+(c)+(d); or
(e)+(f)+(g)+(h).

In another preferred embodiment, X⁴ is arginine, such as D-alanine.

In another preferred embodiment, X⁴ is a non-polar amino acid, preferably selected from the group consisting of:
(a) alanine
(b) glycine
(c) methionine
(d) tryptophan
(e) tyrosine
(f) phenylalanine

In another preferred embodiment, X⁴ is a hydrophobic aliphatic amino acid. Preferably, said hydrophobic amino acid is selected from the group consisting of:
(a) isoleucine
(b) leucine
(c) methionine
(d) phenylalanine
(e) proline
(f) tryptophan
(g) valine
(h) norleucine
(i) homoleucine
(j) homoisoleucine
(k) napthyl alanine
(l) cyclohexyalanine.

In another preferred embodiment, X⁴ is a basic amino acid, preferably selected from the group consisting of:
arginine
histidine
lysine;

In another preferred embodiment, X⁴ is histidine, such as D-histidine.

In another preferred embodiment, X⁴ is a neutral amino acid, preferably selected from the group consisting of:
(a) asparagine
(b) glutamine
(c) threonine
(d) serine
(e) tyrosine

In another preferred embodiment, X⁴ is an acidic amino acid, preferably selected from the group consisting of:
aspartic acid
glutamic acid

In another preferred embodiment, X⁴ is an amino acid comprising a thiol group, such as cysteine.

In another preferred embodiment, X⁴ is a polar amino acid, preferably selected from the group consisting of:
(a) asparagine
(b) glutamine
(c) threonine
(d) serine

In another preferred embodiment, X⁴ is an aromatic amino acid, preferably selected from the group consisting of:
(a) phenylalanine
(b) tryptophan
(c) tyrosine
(d) napthylalanine

In another preferred embodiment, X⁴ is a hydroxy amino acid, such as serine or threonine.

In one preferred embodiment, it is preferred that X⁶ is an alcohol, such as selected from the group consisting of: methanol, ethanol, isopropyl alcohol, ethylene glycol, glycerol and phenol.

In another preferred embodiment, X⁶ is a fatty alcohol, such as selected from the following list:

erucyl alcohol
ricinolyl alcohol
arachidyl alcohol
capryl alcohol
capric alcohol
behenyl alcohol
lauryl alcohol (1-dodecanol)
myristyl alcohol (1-tetradecanol)
cetyl (or palmityl) alcohol (1-hexadecanol)
stearyl alcohol (1-octadecanol)
isostearyl alcohol
oleyl alcohol (cis-9-octadecen-1-ol)
linoleyl alcohol (9Z,12Z-octadecadien-1-ol)
elaidolinoleyl alcohol (9E,12E-octadecadien-1-ol)
linolenyl alcohol (9Z,12Z,15Z-octadecatrien-1-ol)
elaidolinolenyl alcohol (9E,12E,15-E-octadecatrien-1-ol)

In another preferred embodiment, X⁶ is selected from ethanol, glycerol and phenols.

In another preferred embodiment, it is preferred that X⁶ is selected from the group consisting of:

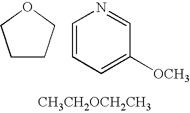

In another preferred embodiment, it is preferred that X⁶ is a hydrocarbon, such as selected from the group consisting of:
aromatic hydrocarbon
saturated hydrocarbon
unsaturated hydrocarbon, such as an alkene, alkyne, or diene

In another preferred embodiment, it is preferred that X⁶ is a saturated hydrocarbon with the length of 1-4 chemical bonds.

In another preferred embodiment, it is preferred that X⁶ is a hydrozaine, such as selected from the group consisting of: hydrazine, 1,1-dimethylhydrazine, and 1,2-dimethylhydrazine.

In another preferred embodiment, it is preferred that X⁶ is a peptide, such as a small peptide with the length of 1 or 2 amino acids.

In another preferred embodiment, X⁶ is an ether, such as dimethylether or methyl ethyl ether.

In another preferred embodiment, it is preferred that X⁶ is a peptidomimetic, such as a small mimetic with the length of 1 or 2 amino acid, such as a peptoid or reduced peptide bond.

In preferred embodiments, X₈ is a hydrogen bond donor, such as an amine or hydroxyl group. In other preferred embodiments, X₈ is NH or SH.

In preferred embodiments, at least two of X¹-X⁵ are D-amino acids. In one preferred embodiment, two of X¹-X⁵ are D-amino acids. In another preferred embodiment, three of X¹-X⁵ are D-amino acids. In another preferred embodiment, four of X¹-X⁵ are D-amino acids. In another preferred embodiment, five of X¹-X⁵ are D-amino acids.

In one preferred embodiment, the GHS-R1A ligand of the present invention has a sequence consisting of SEQ ID NO: 14. In another preferred embodiment, the GHS-R1A ligand of the present invention has a sequence consisting of SEQ ID NO: 15. In another preferred embodiment, the GHS-R1A ligand of the present invention has a sequence consisting of SEQ ID NO: 16. In another preferred embodiment, the GHS-R1A ligand of the present invention has a sequence consisting of SEQ ID NO: 17. In another preferred embodiment, the GHS-R1A ligand of the present invention has a sequence consisting of SEQ ID NO: 18.

GHS-R1A Ligands with Preferred N-Terminal Modifications

In the fourth aspect of the present invention, a GHS-R1A ligand (or a pharmaceutically acceptable salt thereof) is provided,
wherein the GHS-R1A ligand has a structure defined by formula I′″

Z¹-R¹-(X²)-(X³)ₙ-Z², Formula I′″

wherein
Z¹ is an optionally present protecting group;
R1 is selected from:
a) βAla- or
b) βAla-X1- or
c) GABA- or
d) GABA-X1- or
e) Aminopentanoyl-X1- or
f) hydroxy acetic acid (HAA)- or
g) HAA-X1- or
h) a compound with formula A, shown below:

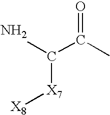

i) a compound with the formula i) shown below:
j) a compound with the formula j) shown below:

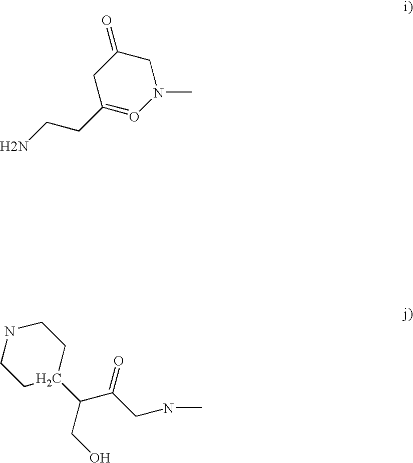

wherein X7 is a spacer with a length of 1-8 chemical bonds, and X8 is a hydrogen bond donor, such as an amine or hydroxyl group;
X¹ is an amino acid selected from naturally occurring and synthetic amino acids;
X² is an anchor group, preferably an amino acid selected from naturally occurring and synthetic amino acids, said amino acid being modified;
each X³ is independently selected from an amino acid, wherein said amino acid is selected from naturally occurring and synthetic amino acids,
wherein X³ optionally may be an anchor group,
Z² is an optionally present protecting group,
n is 0 or an integer in the range of 1-35.

In one preferred embodiment, R1 is a compound with formula A, shown above, wherein X7 preferably has a length of 4-5 chemical bonds. In preferred embodiments, X8 is NH or SH, or, more preferably, an amine or hydroxyl group.

In another preferred embodiment of the present invention, R¹ is selected from:
a) βAla- or
b) βAla-X¹- or
c) GABA- or
d) GABA-X¹- or
e) hydroxy acetic acid (HAA)- or
f) HAA-X¹- or
g) a compound with formula A, shown below:

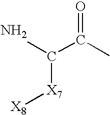

or compounds (I) or (j), above.

In another preferred embodiment of the present invention, R¹ is selected from:
a) βAla- or
b) βAla-X¹- or
c) GABA- or
d) GABA-X¹- or
e) hydroxy acetic acid (HAA)- or
f) HAA-X¹.

In another preferred embodiment of the present invention, R¹ is selected from:
a) βAla- or
b) βAla-X¹- or
e) hydroxy acetic acid (HAA)- or
f) HAA-X¹-

In another preferred embodiment of the present invention, R¹ is selected from:
c) GABA- or
d) GABA-X¹- or
e) hydroxy acetic acid (HAA)- or
f) HAA-X¹-

In one preferred embodiment, X² is selected from the group of modified Ser, modified Cys and modified Lys, such as wherein X² is modified Ser.

In another preferred embodiment, X² is selected from:
a) decenoic acid (L or D form)
b) 5-hexenoic acid (L or D form)
c) 6-heptenoic acid (L or D form)
d) 7-octenoic acid (L or D form)
e) 8-nonenoic acid (L or D form)
f) Ala-3-cp (L or D form)
g) Ala-3-cb (L or D form)
h) Phe-4-Me (L or D form)
i) Phe-4-Et (L or D form)
j) Phe-4-iPr (L or D form)
k) Phe-4-Ph (L or D form)
l) Beta-MeTrp (L or D form)
m) Ala[3-(3-Quinolinyl)] (L or D form)
n) Ala[3-(2-benzimidazoyl)] (L or D form)
o) BenzoTrp (L or D form)
p) 7-AzaTrp (L or D form)
q) Trp(5-NH2) (L or D form);

Other preferred anchor groups for use in the fourth aspect of the present invention include all the embodiments disclosed in the sections entitled “anchor groups” and “Preferred embodiments of X²”, above.

In preferred embodiments of the present invention, the GHS-R1A ligand is selected from one or more of the following:

Z¹-βAla-(X²)-(X³)ₙ-Z², formula II′″

Z¹-βAla-Ser-(X²)-(X³)ₙ-Z², formula III′″

Z¹-GABA-(X²)-(X³)ₙ-Z², formula III′″

Z¹-GABA-Ser-(X²)-(X³)ₙ-Z², formula V′″

Z¹-Aminopentanoyl-Ser-(X²)-(X³)ₙ-Z², formula VII′″

Z¹-HAA-Ser-(X²)-(X³)ₙ-Z², formula VIII′″

Z¹-HAA-(X²)-(X³)ₙ-Z² formula IX′″
wherein Z¹ and Z² are optional protecting groups.

In another preferred embodiment of the present invention, the GHS-R1A ligand is selected from one or more of the following:

Z¹-βAla-(X²)-(X³)ₙ-Z², formula II′″

Z¹-βAla-Ser-(X²)-(X³)ₙ-Z², formula III′″

Z¹-GABA-(X²)-(X³)ₙ-Z², formula IV′″

Z¹-GABA-Ser-(X²)-(X³)ₙ-Z², formula V′″

Z¹-HAA-Ser-(X²)-(X³)ₙ-Z², formula VIII′″

Z¹-HAA-(X²)-(X³)ₙ-Z² formula IX′″
wherein Z¹ and Z² are optional protecting groups.
   (X³)ₙ preferably comprises any of the (X³)ₙ embodiments described under the section entitled “Preferred embodiments of (X³)ₙ”, above.

In one preferred embodiment of the present invention, the compound has SEQ ID NO: 8. In another preferred embodiment, the compound has SEQ ID NO: 9. In another preferred embodiment, the compound has SEQ ID NO: 19.

### Functionality

The GHS-R1A ligands described herein are active at the receptor for GHS as described above, i.e. the receptor GHS-R 1a. The compounds can bind to the receptor, and stimulate, partially stimulate, or inhibit receptor activity. Furthermore, the compounds may be able to modulate the activity of other GHS-R1A ligands, such as ghrelin, by for instance blocking the action of ghrelin—i.e. antagonize the effects of agonists.

Agonists of the GHS-R1A may be either full agonists, i.e. be able to fully stimulate the receptor and the signalling cascades, equal to the activities of ghrelin, or partial agonists, i.e. ligands that are only able to partially stimulate the receptor and the signalling cascade, measured as described below. Such partial agonists may also be able to fully or partially antagonize the actions of full agonists such as ghrelin.

The receptor activity can be measured using different techniques such as detecting a change in the intracellular conformation of the receptor, in the activity of the G-protein coupled to the receptor, and/or in alteration of the level of intracellular messengers.

One simple measure of the ability of a ligand to activate the ghrelin receptor is to measure its EC50, i.e. the dose at which the compound activates the receptor to half of the maximal obtainable effect using same compound. The receptor can either be expressed endogenously on primary cells cultures, for example pituitary cells, or heterologously expressed on cells transfected with a cDNA encoding the ghrelin receptor. Whole cell assays or assays using membranes prepared form either of these cell types can be used depending on the type of assay.

As the receptor is generally believed to be primarily coupled to the Gq signalling pathway, any suitable assay which monitors activity in the Gq/G11 signalling pathway can be used, for example:
1) an assay measuring the activation of Gq/G11 performed for example by measurement of GTPgS binding combined with, e.g., anti-G-alpha-q or -11 antibody precipitation in order to increase the signal to noise ratio. This assay may also detect coupling to other G-proteins than Gq/11.
2) An assay which measure the activity of phopholipase C (PLC) one of the first down-stream effector molecules in the pathway, for example by measuring the accumulation of inositol phosphate which is one of the products of PLC.
3) More down stream in the signalling cascade is the mobilization of calcium from the intracellular stores
4) Further more down stream signalling molecules such as the activity of different kinds of MAP kinases (ERK 1/2, p38, junK, etc.). NF-κ-B translocation and CRE driven gene transcription may also be measured.
5) Binding of fluorescently tagged arrestin to the activated ghrelin receptor

Examples of suitable protocols for use in determining GHS-R1A ligand functionality are given in Example 3.

In one embodiment the binding of a compound to the receptor GHS-R1A is measured by the use of any of the assays described herein above.

A GHS-R1A ligand according to the invention preferably has at least about 50%, at least about 60%, at least about 70%, at least about 80%, or at least about 90%, functional activity relative to 28 aa acylated human ghrelin as determined using the assay described herein above. Greater refers to potency and thus indicates a lesser amount is needed to achieve binding inhibition.

In one embodiment of the invention, the GHS-R1A ligand has a potency (EC50) on the GHS-R 1A of less than 500 nM. In another embodiment the compound has a potency (EC50) on the GHS-R 1A of less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

In a further embodiment the dissociation constant (Kd) of the GHS-R1A ligand is less than 500 nM. In a still further embodiment the dissociation constant (Kd) of the ligand is less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

Binding assays can be performed using recombinantly-produced receptor polypeptides present in different environments. Such environments include, for example, cell extracts and purified cell extracts containing the receptor polypeptide expressed from recombinant nucleic acid or naturally occurring nucleic acid; and also include, for example, the use of a purified GHS receptor polypeptide produced by recombinant means or from naturally occurring nucleic acid which is introduced into a different environment.

Using a recombinantly expressed GHS receptor offers several advantages such as the ability to express the receptor in a defined cell system, so that a response to a compound at the receptor can more readily be differentiated from responses at other receptors. For example, the receptor can be expressed in a cell line such as HEK 293, COS 7, and CHO not normally expressing the receptor by an expression vector, wherein the same cell line without the expression vector can act as a control.

### Protecting Group

Any of the GHS-R1A ligands described herein can comprise a protecting group at the N-terminus or the C-terminus or at both.

A protecting group covalently joined to the N-terminal amino group reduces the reactivity of the amino terminus under in vivo conditions. Amino protecting groups include—C1-10 alkyl, —C1-10 substituted alkyl, —C2-10 alkenyl, —C2-10 substituted alkenyl, aryl, —C1-6 alkyl aryl, —C(O)—(CH2) 1-6-COOH, —C(O)—C1-6 alkyl, —C(O)-aryl, —C(O)—O—C1-6 alkyl, or —C(O)—O-aryl. Preferably, the amino terminus protecting group is acetyl, propyl, succinyl, benzyl, benzyloxycarbonyl or tbutyloxycarbonyl.

A protecting group covalently joined to the C-terminal carboxy group reduces the reactivity of the carboxy terminus under in vivo conditions. The carboxy terminus protecting group is preferably attached to the a-carbonyl group of the last amino acid. Carboxy terminus protecting groups include amide, methylamide, and ethylamide.

### Conjugates

The GHS-R1A ligand of the present invention can be provided in the form of a conjugate, i.e. a molecule comprising the ligand conjugated to another entity.

The other entity may be any substance that is capable of conferring improved properties to the ligand, e.g. in terms of improved stability, half-life, etc.

In one embodiment of the present invention, one or more ligand(s) is conjugated to a polymer molecule. The polymer molecule may be any suitable polymer molecule, such as a natural or synthetic polymer, typically with a molecular weight in the range of about 1-100 kDa, such as about 3-20, kDa, e.g. 5-10 kDa. The polymer is attached to a reactive group present on the GHS-R 1a ligand, e.g. an amine group or a thiol group.

Examples of suitable polymer molecules include polymer molecules selected from the group consisting of polyalkylene oxide (PAO), including polyalkylene glycol (PAG), such as linear or branched polyethylene glycol (PEG) and polypropylene glycol (PPG), poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran. Preferably, the polymer molecule is a PEG molecule, in particular a monofunctional PEG, such as methoxypolyethylene glycol (mPEG). Suitable activated PEG molecules are available from Nektar Therapeutics Inc. (Huntsville Ala., US) or from Valentis, Inc., Burlingame, Calif. U.S.A. Alternatively, the polymer molecules can be activated by conventional methods known in the art, e.g., as disclosed in WO 90/13540. Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which are incorporated herein by reference.

The PEGylation (i.e. conjugation of the ligand and the activated polymer molecule) is conducted in accordance with established procedures, e.g., as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.).

It is also contemplated according to the invention to couple the polymer molecules to one or more GHS-R1A ligands through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378.

In yet another embodiment the GHS-R1A ligand is conjugated to an oligosaccharide molecule, such as dextran, glycan, transferrin, etc. Such conjugation may be achieved in accordance with established technologies, e.g. those available from Neose Technologies, Inc. Horsham, Pa.

In yet another embodiment, the GHS-R1A ligand is conjugated to an Fc region of an IgG molecule, typically in the form of a fusion protein, or to human serum albumin. Methods of such conjugation are known in the art.

Alternatively to providing the GHS-R1A ligand in the form of a conjugate, the GHS-R1A ligand may be modified to include suitable reactive groups, whereby the thus modified GHS-R1A ligand is capable of forming a conjugate in vivo (after having been administered to an individual) through covalent bonding with available reactive functionalities on blood components. The invention also relates to such modified GHS-R1A ligands, and methods for their use. Also, the invention relates to conjugates formed in vitro between a modified GHS-R1A ligand as described above and a blood component. The conjugates formed in accordance with this embodiment are contemplated to have an increased in vivo half life as compared to the corresponding non-modified GHS-R1A ligand.

In accordance with this embodiment, the GHS-R1A ligand is modified with a chemically reactive group (reactive entity). The reactive entity may, e.g., be selected from the wide variety of active carboxyl groups, particularly esters, where the hydroxyl moiety is physiologically acceptable. Such groups may be selected from the group consisting of N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS) and maleimidopropionic acid (MPA). The principal targets for this group of entities are primary amines on the blood component. Another group of active entities is constituted by a maleimido-containing group such as MPA and gamma-maleimide-butrylamide (GMBA) Such groups react with thiol groups present on the blood component. The blood component with which the modified GHS-R1A ligand is designed to react may be any blood component having an available target group, e.g. an amine or a thiol group, and which is suitable as a carrier for binding the modified GHS-R1A ligand in vivo and thereby extend the circulating half-life thereof. Examples of such blood components are serum albumin and IgG.

As mentioned above the covalent bonding of a modified GHS-R1A ligand to a blood component may be achieved in vivo by administration of the modified GHS-R1A ligand directly to the patient by methods as herein described.

### Method for Production

The present invention also relates to a method for production of the GHS-R1A ligands described herein. GHS-R1A ligands can be produced using techniques well known in the art. For example, a polypeptide region of a GHS-R1A ligand can be chemically or biochemically synthesized and modified. Techniques for chemical synthesis of polypeptides are well known in the art. (See e.g., Vincent in Peptide and Protein Drug Delivery, New York, N.Y., Dekker, 1990.) Examples of techniques for biochemical synthesis involving the introduction of a nucleic acid into a cell and expression of nucleic acids are provided in Ausubel, Current Protocols in Molecular Biology, John Wiley, 1987-1998, and Sambrook et al., in Molecular Cloning, A Laboratory Manual, 2 d Edition, Cold Spring Harbor Laboratory Press, 1989. Other examples of methods suitable for use in producing the GHS-R1A ligand of the present invention may be found in WO03084983 (“Process for producing a modified peptide”, Daiichi Suntory Pharma co. Ltd), WO0107475 (“Novel peptides”, Kangawa Kenji) and WO0192292 (“Novel peptides”, Merck & Co. Inc.). Synthesis of proteins and protein chemical modification techniques are also well known in the art, for instance from “Techniques in Protein Modification” by Roger L. Lundblad (CRC-Press); “Chemical Approaches to the Synthesis of Peptides and Proteins” by Paul Lloyd-Williams et al. (CRC-Press); “Chemical Reagents for Protein Modification” by Roger L. Lundblad (CRC Press); furthermore organic groups for modification onto the ligands of the present invention may be synthesised using organic synthesis methods well understood by one skilled in the art, e.g. “Side Reactions in Organic Synthesis: A Guide to Successful Synthesis Design”, by Florencio Zaragoza Dörwald (John Wiley and Sons); “Modern Methods of Organic Synthesis” 2ed (Cambridge Texts in Chemistry and Biochemistry) by W. Carruthers; “Lipid Modifications of Proteins”, Methods in Enzymology, Volume 250, Elsevier Science.

### Pharmaceutical Composition

While it is possible for a GHS-R 1a ligand or salt thereof of the present invention to be administered as the raw chemical, it is preferred to present it in the form of a pharmaceutical composition. Accordingly, in one aspect the present invention relates to a pharmaceutical composition comprising a GHS-R1A ligand (or pharmaceutically acceptable salt thereof) according to the present invention. The pharmaceutical composition of the present invention preferably comprises a pharmaceutically acceptable carrier, vehicle and/or excipient. The carrier, vehicle and/or excipient should be compatible with the GHS-R 1a ligand or salt thereof. In a preferred embodiment, the pharmaceutical composition is not immunogenic when administered to a human in accordance with the present invention.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

Suitable pharmaceutical carriers include sterile aqueous solution and various organic solvents and inert solid diluents or fillers. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Suitable excipients are, for example, water, saline, dextrose, glycerol, ethanol or the like and combinations thereof.

In addition, if desired, the composition can contain minor amounts of auxiliary substances such as wetting or emulsifying agents, pH buffering agents and the like which enhance the effectiveness of the active ingredient. It is preferred that the formulation has a pH within the range of 3.5-8, such as in the range 4.5-7.5, such as in the range 5.5-7, such as in the range 6-7.5, most preferably around 7.3. However, as is understood by one skilled in the art, the pH range may be adjusted according to the individual treated and the administration procedure. For example, some GHS-R 1a ligands may be easily stabilised at a lower pH, so in another preferred embodiment of the invention the formulation has a pH within the range 3.5-7, such as 4-6, such as 5-6, such as 5.3-5.7, such as 5.5.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

The pharmaceutical composition of the present invention can include a pharmaceutically acceptable salt of the GHS-R 1a ligand therein. The salt will be one which is acceptable in its therapeutic use. By that it is meant that the salt will retain the biological activity of the GHS-R 1a ligand and the salt will not have untoward or deleterious effects in its application and use in treating diseases.

Pharmaceutically acceptable salts are prepared in a standard manner. If the GHS-R 1a ligand is a base it is treated with an excess of an organic or inorganic acid in a suitable solvent. If the GHS-R 1a ligand is an acid, it is treated with an inorganic or organic base in a suitable solvent.

The pharmaceutically acceptable salt may be an acid addition salts including salts of inorganic acids as well as organic acids. Acid addition salts are formed with free amino groups of the GHS-R 1a ligand. Representative examples of suitable inorganic acids include hydrochloric, hydrobromic, hydriodic, metaphosphoric, phosphoric, sulpfuric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, ethylenediaminetetraacetic (EDTA), p-aminobenzoic, glutamic, benzenesulfonic and ptoluenesulfonic acids and the like. Further examples of pharmaceutically acceptable inorganic or organic acid addition salts include the pharmaceutical acceptable salts listed in J. Pharm. Sci. 1977, 66, 2, which is incorporated herein by reference. The metal salt may be an alkali metal or earth alkali metal salt. Examples of metal salts include lithium, sodium, potassium and magnesium salts and the like. Examples of ammonium and alkylated ammonium salts include ammonium, methylammonium, dimethylammonium, trimethylammonium, ethylammonium, hydroxyethylammonium, diethylammonium, butylammonium and tetramethylammonium salts and the like.

Salts formed with the free carboxyl groups can be derived from inorganic bases such as, for example, sodium, potassium, ammonium, calcium or ferric hydroxides, and such organic bases as isopropylamine, trimethylamine, 2-ethylamino ethanol, histidine, procaine and the like.

Also included within the scope of pharmaceutical acceptable acid addition salts of a GHS-R 1a ligand of the invention is any hydrate (hydrated form) thereof.

The pharmaceutical composition of the invention may further comprise transport molecules. Transport molecules are primarily added in order to increase the half-life of the a GHS-R 1a ligand of the invention comprising an anchor group, by preventing premature cleavage of the anchor group or part thereof from the amino acid backbone of the ligand. Transport molecules act by having incorporated into or anchored to it a GHS-R1A ligand according to the invention.

Any suitable transport molecules known to the skilled person may be used. Examples of transport molecules are those described in the conjugate section. Other preferred examples are liposomes, micelles, and/or microspheres.

Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipids for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipid or modifiers of liposomes are preferred e.g. in combination with cholesterol and/or phosphatidylcholine.

Long-circulating liposomes are characterized by their ability to extravasate at body sites where the permeability of the vascular wall is increased. The most popular way to produce long circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipid applicable for liposomes are supplied by Avanti, Polar lipids, Inc, Alabaster, Ala. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. One method is described in example 5. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

Micelles are formed by surfactants (molecules that contain a hydrophobic portion and one or more ionic or otherwise strongly hydrophilic groups) in aqueous solution. As the concentration of a solid surfactant increases, its monolayers adsorbed at the air/water or glass/water interfaces become so tightly packed that further occupancy requires excessive compression of the surfactant molecules already in the two monolayers. Further increments in the amount of dissolved surfactant beyond that concentration cause amounts equivalent to the new molecules to aggregate into micelles. This process begins at a characteristic concentration called “critical micelle concentration”.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In a preferred embodiment of the invention the composition of the invention comprises a GHS-R 1a ligand or a salt thereof, as a lyophilisate and a solvent, said lyophilisate and said solvent being in separate compartements until administration. In another embodiment the composition is a solution of the GHS-R1A ligand or a salt thereof. In both embodiments the solvent may be any suitable solvent, such as described herein, and preferably the solvent is saline.

The invention also relates to a method for preparing a medicament or pharmaceutical composition comprising a compound of the invention, comprising admixing at least one GHS-R1A ligand or a salt thereof the present invention with a physiologically acceptable carrier.

In one aspect the invention relates to a pharmaceutical composition comprising at least one GHS-R1A ligand of the present invention, or a pharmaceutically acceptable salt thereof. In a preferred embodiment the pharmaceutical composition comprises at least two different GHS-R1A ligands of the present invention or pharmaceutically acceptable salt(s) thereof. The difference may for example be compounds having different anchor groups.

In one embodiment of the present invention, said pharmaceutical composition further comprises human ghrelin or an analogue thereof (or a respective pharmaceutically acceptable salt thereof).

In another preferred embodiment, the pharmaceutical composition comprises at least one GHS-R1A ligand according to the present invention, or a pharmaceutically acceptable salt thereof, in combination with a desacylated Ghrelin-like compound, or a pharmaceutically acceptable salt thereof, such as any of the desacylated ghrelin-like compounds described in WO03051389 (Theratechnologies: “Pharmaceutical compositions comprising unacylated ghrelin and therapeutical uses thereof”)

### Compositions for Parenteral Administration

The GHS-R 1a ligand or a salt thereof of the present invention may be formulated for parenteral administration (e.g., by injection, for example bolus injection or continuous infusion) and may be presented in unit dose form in ampoules, pre-filled syringes, small volume infusion or in multi-dose containers with an added preservative. A pharmaceutical composition for parenteral administration may include sterile aqueous and non-aqueous injectable solutions, dispersions, suspensions or emulsions in oily or aqueous vehicles, for example solutions in aqueous polyethylene glycol, as well as sterile powders to be reconstituted in sterile injectable solutions or dispersions prior to use.

The active ingredient may be in powder form, obtained by aseptic isolation of sterile solid or by lyophilisation from solution for constitution before use with a suitable vehicle, e.g., sterile, pyrogen-free water. Aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions of GHS-R1A ligands or pharmaceutically acceptable salts thereof can be prepared in water or saline, and optionally mixed with a nontoxic surfactant. Compositions for intravenous or intra-arterial administration may include sterile aqueous solutions that may also contain buffers, liposomes, diluents and other suitable additives.

Examples of oily or nonaqueous carriers, diluents, solvents or vehicles for parental use include propylene glycol, polyethylene glycol, animal, synthetic or vegetable oils, and injectable organic esters, and may contain formulatory agents such as preserving, wetting, emulsifying or suspending, stabilizing and/or dispersing agents. Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Suitable organic esters include fatty acid esters such as ethyl oleate and isopropyl myristate.

Suitable soaps for use in parenteral compositions include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating the compound(s) or pharmaceutically acceptable salt(s) thereof in the required amount in the appropriate solvent with various of the other ingredients enumerated above, as required, followed by filter sterilization.

### Compositions for Oral Delivery

Those GHS-R1A ligand types capable of remaining biologically active in an individual after oral administration (such as short peptides) can be formulated in a wide range of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably contain 1-70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the composition of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Other forms suitable for oral administration include toothpaste, gel dentrifrice orchewing gum. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, emulsions, syrups and elixirs and may contain, in addition to the active component, colorants, flavors, stabilizers, buffers, artificial and natural sweeteners, dispersants, thickeners, solubilizing agents, and the like.

### Compositions for Topical Administration

The compounds of the invention can also be delivered topically. Regions for topical administration include the skin surface. Compositions for topical administration via the skin and mucous membranes should not give rise to signs of irritation, such as swelling or redness.

The compounds described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

The compounds of the present invention may be formulated for topical administration to the epidermis as ointments, creams, gels or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Compositions suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

### Compositions for Aerosol, Nasal or Inhalation Delivery

The compounds of the present invention may be formulated for administration to the respiratory tract and including intranasal administration.

The compounds of the present invention may also be formulated for nasal administration. The solutions or suspensions are applied directly to the nasal cavity by conventional means, for example with a dropper, pipette or spray. The compositions may be provided in a single or multidose form. In the latter case of a dropper or pipette this may be achieved by the patient administering an appropriate, predetermined volume of the solution or suspension. In the case of a spray this may be achieved for example by means of a metering atomizing spray pump. A suitable formulation for nasal administration is described in EP 1 466 610.

For inhalation, the compounds of the present invention can be formulated as using methods known to those skilled in the art, for example an aerosol, dry powder or solubolized such as in microdroblets, preferably in a device intended for such delivery (such as commercially available from Aradigm, Alkerme or Nektar).

Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents in accordance with methods known in the art.

### Administration as Suppositories

The compounds of the present invention may also be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

### Compositions for Other Types of Delivery

Other types of delivery of the compounds according to the present invention are also foreseen, such as implants.

### Administration

Suitable dosing regimens for the various compounds and methods of the present invention are preferably determined taking into account factors well known in the art including type of subject being dosed; age, weight, sex and medical condition of the subject; the route of administration; the renal and hepatic function of the subject; the desired effect; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug.

The present invention preferably deals with methods for administering a GHS-R1A ligand in a way which mimics the physiologically pre-meal situation as closely as possible yet providing patients in need of increased food intake, for example fragile elderly, post operative patients, patients with lost appetite as part of cachexia for example precipitated by cancer, cardiac disease etc. with a sufficient extra stimulatory input to their appetite regulating ghrelin receptors, which normally are reached by ghrelin in the pre-meal situation.

A typical dosage of a compound employed according to the invention (irrespective of administration route) is in a concentration equivalent to from 10 ng to 10 mg ghrelin per kg bodyweight. The dose level is based on the activity and the pharmacokinetic profile of the compound, i.e. the intrinsic in vitro activity relative to acylated ghrelin and the plasma curve observed in patients after administration of the compound. The selected dose is given to ensure that the resulting area under the activity curve is equivalent to the area under the ghrelin activity curve. The concentrations and amounts herein are given in equivalents of amount ghrelin, wherein the ghrelin is the 28 aa human acylated ghrelin. The activity curve is defined as the plasma concentration curve multiplied by the intrinsic activity of the compound as measured using the methods described in the section entitled “Functionality”, above.

In a preferred embodiment the medicament is administered in a concentration equivalent to from 0.1 μg to 1 mg ghrelin per kg bodyweight, such as from 0.5 μg to 0.5 mg ghrelin per kg bodyweight, such as from 1.0 μg to 0.1 mg ghrelin per kg bodyweight, such as from 1.0 μg to 50 μg ghrelin per kg bodyweight, such as from 1.0 μg to 10 μg ghrelin per kg bodyweight.

The administration route used must ensure that the non-degraded, bioactive form of the ligand will be the dominating form in the circulation, which will reach the GHS-R1a receptors and stimulate these. Thus, in order to obtain the maximum effect of the medicament it is preferably administered from one to three times daily, each administration being within 45 minutes of a meal, such as within 30 minutes of a meal, such as within 25 minutes of a meal, such as within 20 minutes of a meal, such as within 15 minutes of a meal, such as within 10 minutes of a meal, such as within 5 minutes of a meal. More preferred the medicament is administered prior to each main meal, such as administered three times daily. This administration scheme is applicable for all of the administrations route mentioned herein.

The compound according to the present invention may be administered in any suitable form, such as one or more of the following administration forms: oral, nasal, parenteral, including subcutaneously, intraveneously and intramuscularly, topical, buccal, sublingual, transdermal, inhalation, needle-free or in the form of a suppository.

### Subcutaneous Administration

Any parenteral administration form that will ensure that the ghrelin receptors which normally are the target for peripherally produced ghrelin in the premeal situation will be exposed to sufficient levels of the bioactive form of a GHS-R 1a ligand or a salt thereof to ensure robust and appropriate appetite stimulation, without causing desensitization of the system, may be part of the present invention. However, taken into consideration that the individuals to be treated possibly will have to receive treatment for a longer period, such as weeks or months, it is preferred that the administration form is well suited herefor.

Accordingly, it is preferred that the GHS-R1A ligand or a salt thereof according to the invention is administered subcutaneously in an amount sufficient to allow sufficient levels of the bioactive form, e.g. an acylated or anchorgroup containing form, to reach the receptors in time, such as prior to the forthcoming meal. Dose and frequency of administration by the subcutaneous route are as described above.

### Bolus Administration

Furthermore, from a molecular pharmacological point-of-view it is important to note that it has been found that the ghrelin receptor normally is exposed to short-lived surges in the concentrations of the natural agonist ligand, ghrelin. The GHS-R 1a receptor belongs to the class of receptors, so-called G protein coupled receptors or 7TM receptors, that upon continued exposure to an agonist will be desensitizised, internalized and down-regulated. These mechanisms, which are inherent to the overall signal transduction system, involve processes such as receptor phosphorylation (which in itself decreases the affinity of the receptor for the agonist) binding of inhibitory proteins such as arrestin (which sterically block the binding of signal transduction molecules such as G proteins). Another part of the agonist mediated desensitization process is receptor internalization (i.e. physical removal of the receptor from the cell surface where it could bind the agonist) as well as receptor down regulation (i.e. decreased production/expression of the receptor). Receptor internalization could after short-lived exposure of the receptor to agonist be followed by a re-sensitization process, where the receptor is dephosphorylated and recycled to the cell surface to be used again. Without being bound by theory, it is believed that, upon prolonged stimulation, which would occur for example during a long-lasting continuous infusion of the agonist, the receptor down-regulation process ensures that the target cell is adjusted in its signal transduction system etc. to this situation.

The present invention provides a procedure for an optimal administration of a GHS-R1A ligand to patients in order to obtain a maximal response and avoid for example desensitization mechanisms.

Accordingly, the present invention relates in one aspect to administration of a GHS-R1A ligand in boluses, preferably a bolus prior to each main meal. It has been found that a bolus administration leads not only to stimulation of appetite, but also to stimulation of feed intake and to stimulation of weight gain or maintenance of weight.

Without being bound by theory, it is believed that premeal subcutaneous injection, intravenous injection or short-term infusions of appropriate doses of a GHS-R1A ligand will ensure that a robust stimulation of appetite inducing GHS-1A receptors will be obtained with minimal constraint to the mobility etc. of the patient. Thus for example, patients with hip fractures can in the post operative situation be treated in the premeal period and if required during the meal as such, but will be free to move around and participate in the important post operative physicotherapeutic regimens.

Bolus administration is particularly relevant for increasing weight, fat mass and/or appetite in an individual suffering from cachexia, such as cancer cachexia.

In one embodiment the medicament is administered by a suitable administration route as a bolus prior to a meal, said bolus comprising an amount of the GHS-R1A ligand or a salt thereof equivalent to from 0.3 μg to 600 mg ghrelin. More preferably, the medicament is administered as a bolus prior to a meal, said bolus comprising an amount of the GHS-R1A ligand or a salt thereof equivalent to from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin. In one preferred embodiment of the present invention, the GHS-R1A ligand according to the present invention is administered as a bolus in an amount equivalent to 10 μg per kg body weight or 2×10 μg per kg body weight. The exact dose depend, e.g. on the bioavailability of the composition in question, the higher the bioavailability the lower the dose.

### Indications

Throughout the application, including the present section, it will be understood that whenever GHS-R 1a ligands are mentioned as useful for a specific indication, the term GHS-R1a ligand encompasses GHS-R 1a ligand as such as well as a salt, hydrate or any other derivative thereof, optionally in the form of a pharmaceutical composition as described herein.

The present invention relates to use of one or more of the GHS-R1A ligands according to the present invention in the manufacture of a medicament for the treatment of an individual in need thereof. Preferably, said individual is suffering from, or at risk of suffering from, a pathological condition treatable with the GHS-R1A ligands of the present invention. The present invention also relates to a method of treatment of a individual in need thereof, comprising administering to said individual one or more of the GHS-R1A ligands according to the present invention.

Thus, the GHS-R1A ligands of the present invention may be used to treat any individual capable of receiving benefit from said treatment. In one preferred embodiment, one or more of the GHS-R1A ligands of the present invention are used for any of the following:

In one aspect of the invention the GHS-R1A ligand is a GHS-R1A agonist.

The GHS-R1A agonist may be used in the treatment or prevention of one or more of the following conditions and diseases discussed below.

In one embodiment the GHS-R1A ligand may be used for prevention and treatment of cachexia/malnutrition or maintenance of metabolic homeostasis in patients.

The word cachexia comes from the Greek kakos for “bad” and hexis for “condition.” Cachexia, or wasting, is one of the most distressing and devastating symptoms of several severe diseases, robbing people of their energy, sense of well-being, and quality of life, and increasing their dependence on others.

The foremost sign of cachexia is weight loss, not only of fatty tissue but also of muscle tissue and even bone. This non-fatty tissue is also known as “lean body mass.” In addition, there is loss of appetite (anorexia), weakness (asthenia), and a drop in hemoglobin level (anemia).

Treatment of cachexia is not simply a matter of eating more. Even if the person wants to eat, even if he or she tries to eat, even if the person is given nutrients through a stomach tube or intravenously, the condition will normally not be reversed.

Recent research has revealed that the condition is now regarded as part of the body's reaction to the presence of the underlying disease. Recent research also indicates that, in some cases, tumors themselves produce substances that induce cachexia.

Thus, in one aspect, the invention relates to the use of a GHS-R1A ligand disclosed herein for the preparation of a medicament for

a) stimulation of appetite, and/or
b) stimulation of food intake, and/or
c) stimulation of weight gain, and/or
d) increasing body fat mass, and/or
e) increasing lean body mass.

In particular the GHS-R1A ligand may be used in the prevention and treatment of cachexia or malnutrition in individuals suffering from:
Cancer
AIDS
Cardiac failure
Pulmonary insufficiency due to for instance COPD, pulmonary fibrosis, cystic fibrosis or alpha-1-antitrypsin deficiency
Renal failure due to for instance glomerulonephritis, tubulointerstitial nephropathy, hereditary nephropathy (e.g. polycystic kidney disease), hypertension, diabetes mellitus, vascular disease, obstructive uropathy
Liver failure due to for instance viral hepatitis, toxic hepatitis, fibrosis, cirrhosis, primary biliary cirrhosis, hereditary liver diseases
Autoimmune diseases such as rheumatoid arthritis, systemic lupus erythematosus
Severe chronic infections such as tuberculosis
Patients undergoing gastrectomy
Organ transplantation patients
Patients recovering from major surgery
Patients recovering from trauma, such as especially trauma requiring surgical intervention
Premature children and children born small for gestastional age

It is also preferred that the GHS-R1A ligand according to the invention may be administered prophylactically for preventing a cachectic state. Thus, in addition to the treatment of cachexia and malnutrition, the GHS-R1A ligands according to the present invention may be useful in prevention or treatment of a catabolic state, such as a catabolic state resulting from:
Critical illness
Severe infections such as sepsis
Severe burns
Major trauma
Major surgery such as gastrointestinal surgery, cardiothoracic surgery, transplantation
Treatment with catabolic agents such as glucocorticoids

Due to the positive effect of the GHS-R1A ligands on quality of life (QOL), for example as is caused by improved appetite and/or body weight and/or nutritional status, the GHS-R1A ligands according to the invention are suitable for prevention and treatment of frailty in elderly persons.

In another embodiment the GHS-R1A ligand according to the invention is useful in the prevention or treatment of heart failure. In particular the heart failure may be due to ischaemic heart disease, cardiomyopathy, hypertension, valvular dysfunction or heart failure due to pulmonary disease such as chronic bronchitis, pulmonary hypertension, emphysema or heart failure caused by genetic defects.

Furthermore, the GHS-R1A ligand according to the invention is useful in prevention or treatment of tissue ischaemia. For example the ischaemia may be cardiac ischaemia due to for instance coronary artery disease, or cerebral ischaemia due to thrombo-embolism

Furthermore, the GHS-R1A ligands according to the invention are suitable for prevention and/or treatment of bone and cartilage related diseases. In particular the GHS-R1A ligands are useful in the prevention or treatment of osteoporosis.

Also, the GHS-R1A ligands according to the invention are suitable in the treatment of bone fractures, by acceleration of fracture healing and recovery following major fractures.

The GHS-R1A ligands of the present invention also improve the nutritional condition of the individuals being treated due to an increase of gastric motility and gastric emptying. Accordingly, the GHS-R1A ligand of the present invention may be used in the treatment or prevention of delayed gastric emptying/gastroparesis, such as in patients with diabetes mellitus, patients with renal failure patients with liver failure, patients with idiopathic gastroparesis, critically ill patients, patients undergoing anaestesia and patients undergoing surgery.

Furthermore, the GHS-R1A ligands may be used for prevention and treatment of postoperative ileus.

In a further embodiment the GHS-R1A ligands are useful in the prevention and treatment of inflammatory diseases, such as inflammatory bowel diseases.

In a further embodiment the GHS-R1A ligand according to the invention is useful in the treatment of malignant diseases, such as breast cancer and thyroid cancer.

In a further embodiment the GHS-R1A ligand according to the invention is useful in the treatment of hyperthyroidism, thus, GHS-R1A ligands according to the invention may be used for preventing weight gain in individuals being converted from a hyperthyroidic state to euthyroid state.

The GHS-R1A ligands according to the invention are also useful in the treatment of sleeping disorders, and accordingly, the GHS-R1A ligands may be used for treating sleeping disorders.

In yet another embodiment, the invention relates to use of a GHS-R1A ligand in the treatment of a lipodystrophic syndrome, or for the manufacture of a medicament for the prevention or treatment of a lipodystrophic syndrome. Lipodystrophic syndromes encompass a heterogeneous group of rare disorders characterized by partial or generalized loss of adipose tissue depots. Some patients may have only cosmetic problems while others may also have severe metabolic complications such as dyslipidemia, hepatic steatosis, and severe insulin resistance. These disorders can either be inherited (familial or genetic lipodystrophies) or can occur secondary to various types of illnesses or drugs (acquired lipodystrophies).

In another aspect of the invention the GHS-R1A ligand is a GHS-R1A antagonist, a GHS-R1A inverse agonists, or a GHS-R1A partial agonist. Any such compound can be used to inhibit one or more of the actions of ghrelin. Such inhibition may be useful to treat a number of diseases, syndromes and states, such as conditions associated with or caused by relatively increased food intake.

A GHS-R1A antagonist, a GHS-R1A inverse agonists, or a GHS-R1A partial agonist according to the invention is in particular suitable in prevention and treatment of obesity, such as for reducing weight, and maintaining weight. Furthermore, these compounds may be used in the treatment of eating disorders such as binge eating, night eating or craving.

GHS-R1 antagonists and partial antagonists may also be used for prevention and/or treatment of diabetes.

### Combination Treatments

In a further aspect of the invention the present compounds may be administered in combination with further pharmacologically active substances or therapeutic method or other pharmacologically active material, By the phrase “in combination” with another substance(s) and/or therapeutic method(s) is meant herein that said another substance(s) and/or therapeutic method(s) is administered to the individual thus treated before, during (including concurrently with) and/or after treatment of an individual with a GHS-R1A ligand. In all cases of combination treatment described herein, the combination can be in the form of kit-in-part systems, wherein the combined active substances may be used for simultaneous, sequential or separate administration. In all cases, it is preferred that any of the herein-mentioned medicaments are administered in pharmaceutically effective amounts, i.e. an administration involving a total amount of each active component of the medicament or pharmaceutical composition or method that is sufficient to show a meaningful patient benefit.

In the following sections, combination therapies for use in preferred embodiments of the present invention are grouped as follows:
1) Combinations wherein all active ingredients are appetite-regulating agents or in other ways useful for treating cachexia
2) Combinations of the GHS-R1A ligand of the present invention with an ingredient or therapy active against a disease causing or being associated with the disease or condition treated with the GHS-R1A ligand.
3) Combinations of the GHS-R1A ligand with an ingredient active or therapy against symptoms associated with the disease or condition treated with the secretagogue such as ghrelin or a GHS-R1A ligand.

Of course, combinations of the above groups are also within the scope of this invention.

Combinations Wherein all Active Ingredients are Appetite-Regulating Agents or in Other Ways Useful for Treating Cachexia and/or Lipodystrophy

The GHS-R1A ligand (s) according to the invention can be administered in combination with other appetite-regulating agents, including more than one type of growth hormone secretagogue, such as a GHS-R1A ligand or ghrelin itself. Other secretagogues suitable for combination administration with another secretagogue compound are any of the GHS-R1A ligands described herein. In one preferred embodiment of the present invention, wild type ghrelin (most preferably human wild type ghrelin) is administered in combination with one or more of the GHS-R1A ligands of the present invention—this combination is envisaged to enhance and/or prolong the effect of the ligands on the ghrelin receptor. In another preferred embodiment of the present invention, a GHS-R1A ligand that is not wild type ghrelin is administered in combination with a GHS-R1A ligand of the present invention: this combination is envisaged to enhance and/or prolong the effect of the ligands on the GHS-1A receptor. In a similar way, several different GHS-1A ligands (including at least one type of GHS-R1A ligand according to the present invention) may be administered to an individual to increase efficacy on the ghrelin receptor—such as greater than 2 different ligand types, such as 3, such as 4, such as 5, such as 6, such as 7, such as greater than 8 different ligand types. The GHS-R1A ligand according to the invention can also be administered in combination with a pharmaceutically effective amount of a growth hormone, including hGH.

In one preferred embodiment of the present invention the GHS-R1A ligand of the present invention may be administered in combination with IGF-1, IGFBP-3, or ALS, preferably with IGF-1. The rationale behind this combination treatment is to increase the level of IGF-1, IGFBP-3, and/or ALS found to be low in e.g. cachectic individuals.

In a further embodiment of the invention, the GHS-R1A ligand (s) of the present invention can be administered in combination with compounds known to stimulate appetite, such as melanocortin receptor antagonists, neuropeptide Y receptor agonists including agonists selective for individual subtypes of the neuropeptide Y receptors, leptin or leptin receptor agonists, cannabinoids including marijuana and marijuana derivatives, antipsychotics, especially atypical antipsychotics such as sertindole, Sulpirid, Clozapine, Risperidone, Quetiapin, Amisulpride, Ziprasidon, and Olanzapine.

Combinations of the GHS-R1A Ligand with an Ingredient or Therapy Active Against a Disease Causing or being Associated with the Disease or Condition Treated with the GHS-R1A Ligand

In particular in relation to cancer cachexia, administration of a GHS-R1A ligand of the present invention may be used in combination with any anti-cancer therapy, including antineoplastic chemotherapy, radiotherapy and surgical treatment. In particular it is used in combination with chemotherapy and radiotherapy. Thus, in one embodiment the present invention relates to a method of treating cancer comprising administering an effective amount of radiotherapy and an effective amount of a GHS-R1A ligand of the present invention. The treatment with the GHS-R1A ligand may be started before the radiotherapy treatment initiates. The analogue may be administered continuously (e.g. during the radiotherapy) or it may be administered at intervals, for example between periods with radiotherapy therapy.

In another embodiment the present invention relates to a method of treating cancer comprising administering an effective amount of antineoplastic chemotherapy and an effective amount of a GHS-R1A ligand of the present invention. The treatment with the GHS-R1A ligand may be started before the chemotherapy treatment initiates. It may be administered continuously during the chemotherapy or it may be administered at intervals, for example between periods with chemotherapy therapy.

Furthermore, the combination treatment may be co-formulations of the GHS-R1A ligand and the antineoplastic chemotherapy.

The GHS-R1A ligand according to the invention can also be administered in combination with a pharmaceutically effective amount of glucocorticoid steroids and prokinetic treatment as well as other treatment used in cancer therapy. Thus, in another preferred embodiment of the present invention, the GHS-R1A ligand according to the invention is administered in combination with a pharmaceutically effective amount of one or more of:

progestational drugs, such as megastrol and/or
cyproheptadines (and/or other 5-HT receptor antagonists), and/or
branched chain amino acids, and/or
oxandralin and/or
anti-TNF-alpha agents, such as infliximab, etanercept, or adalimumab and/or testosterone and/or
“cocktail” comprising immunonutrition, antioxidants and COX-2 inhibitors and/or
cannabinoids, and/or
eicosapentaenoic acid and/or
melatonin and/or
thalidomide and/or
a β₂ adrenergic drug; most preferably for the treatment of cachexia, such as cancer cachexia.

In yet another embodiment the GHS-R1A ligand according to the invention is administered in combination with anti-inflammatory compounds, preferably an NSAID, such as indomethacin, and COX1 inhibitors or COX2 inhibitors, and/or anti-TNF-alpha compounds such as infliximab, etanercept, or adalimumab. Another combination may be with erythropoietin/EPO. Another combination can be with angiotensin II lowering agents, such as Vitor. Another combination can be with selective androgen receptor modulator(s). Another combination may be with one or more of leptin, agonists of the renin-angiotensin system, opioid receptor agonists or peroxisome proliferator-activated receptor gamma agonists.

In relation to treatment of lipodystrophy, the invention relates in another embodiment to a treatment wherein a GHS-R1A ligand according to the present invention is administered in combination with a lipodystrophy treatment, such as one or more of the treatments or compounds described herein suitable for treating a lipodystrophic syndrome.

Thus, other pharmacologically active substances that may be administered in combination with the GHS-R1A ligand of the present invention, in the methods of the present invention comprise:
a) Leptin: leptin has been shown to have a positive effect on the metabolic abnormalities associated with lipodystrophy. This treatment has proven to be beneficial both to those patients that suffer from a low plasma level of leptin and to those that have a normal level.
b) Peroxisome proliferator-activated receptor (PPAR-γ) agoinsts: PPAR-γ has in several studies been demonstrated to be important for adipocyte metabolism and metabolic syndrome and it is proposed that PPAR-γ agonists will decrease the symptoms of lipodystrophy.
c) Agonists of the renin-angiotensin system: it has been shown that treatment with HAART increases the activity of ACE in the T-cells, which means that agonists of the rennin-angiotensin system may improve HAART induced lipodystrophy.
d) Opioid receptor antagonists: opioid receptor antagonists, such as Naloxone and Naltrexone, have been shown to prolong the period of time from protease inhibitor treatment to development of the first symptoms of lipodystrophy.
e) Des-acyl ghrelin: for example, ghrelin in combination with des-acyl ghrelin have been found to decrease insulin resistance, which is an important feature of the lipodystrophy syndrome.
f) Adiponectin and anti-diabetic treatment including other compounds for the treatment and/or prevention of insulin resistance and diseases wherein insulin resistance is the pathophysiological mechanism.
g) Therapy with rhGH has been reported to cause reduction in the size of ‘buffalo hump’, truncal fat and to increase the lean body mass in a small number of patients. However, fat loss and lipid abnormalities did not improve and blood glucose control worsened. Examples of syndromes treated with hGH include HIV, AIDS and cancer. Without being bound by theory, it is believed that treatment with the ligand(s) of the present invention would maintain and/or increase body fat in patients being treated with hGH, thereby effectively counteracting or at least reducing lipodystrophy caused by hGH. Thus, in one preferred embodiment, the present invention relates to use of a ligand according to the present invention in combination with a growth hormone, preferably in individuals suffering from HIV or AIDS and/or cancer cachexia. Said treatment with a ligand may be prior to, and/or during and/or after the individual is subjected to treatment with a growth hormone. Said growth hormone is preferably hGH.
h) Treatment with combinations of different secretagogues as described herein.
   Combinations of the GHS-R 1A Ligand with an Ingredient Active or Therapy Against Symptoms Associated with the Disease or Condition Treated with the GHS-R1A Ligand

The invention further relates to combination treatment, wherein one of the ingredients in the combination is used for treating symptoms or conditions that may be encountered in individuals suffering from cachexia. Thus, uses and combination treatments involving administration of a GHS-R1A ligand according to the present invention, can also involve treatment in combination with one or more of:

a) prophylaxis and/or alleviation and/or treatment of a clinical depression, which combination treatment further comprises administering an antidepressant, a prodrug thereof, or a pharmaceutically acceptable salt of said antidepressant or said prodrug. In the above combination treatment, the antidepressant is preferably a norepinephrine reuptake inhibitor (NERI), a selective serotonin reuptake inhibitor (SSRI), a monoamine oxidase inhibitor (MAO), a combined NERI/SSRI, or an atypical antidepressant, a prodrug of said antidepressant or a pharmaceutically acceptable salt of said antidepressant or said prodrug.

One preferred antidepressant is a selective serotonin reuptake inhibitor (SSRI), a prodrug thereof or a pharmaceutically acceptable salt of said SSRI or said prodrug. The SSRI is preferably citalopram, escitalopram, femoxetine, fluoxetine, fluvoxamine, indalpine, indeloxazine, milnacipran, paroxetine, sertraline, sibutramine or zimeldine, a prodrug of said SSRI or a pharmaceutically acceptable salt of said SSRI or said prodrug. Of the above, citalopram and escitalopram, a prodrug or a pharmaceutically acceptable salt thereof, are preferred in certain embodiments of combination treatments according to the present invention.

c) prophylaxis and/or alleviation and/or treatment of a psychotic condition, which combination treatment further comprises administering an antipsychotic agent, a prodrug thereof or a pharmaceutically acceptable salt of said antipsychotic agent or said prodrug Preferred antipsychotic agents used in combination treatments in accordance with the present invention include chlorpromazine, haloperidol, clozapine, loxapine, molindone hydrochloride, thiothixene, olanzapine, ziprasidone, ziprasidone hydrochloride, prochlorperazine, perphenazine, trifluoperazine hydrochloride and risperidone.
d) prophylaxis and/or alleviation and/or treatment of anxiety, which combination treatment further comprises administering an antianxiety agent, a prodrug thereof or a pharmaceutically acceptable salt of said antianxiety agent or said prodrug. Preferred antianxiety agents used in combination treatments in accordance with the invention include alprazolam, clonazepam, lorazepam, oxazepam, chlordiazepoxide hydrochloride, diazepam, buspirone hydrochloride, doxepin hydrochloride, hydroxyzine pamoate and clonazepam.

### Medical Packaging

The compounds of the invention may be administered alone or in combination with pharmaceutically acceptable carriers or excipients, in either single or multiple doses. The formulations may conveniently be presented in unit dosage form by methods known to those skilled in the art.

It is preferred that one or more of the compounds according to the invention are provided in a kit. Such a kit typically contains an active compound in dosage forms for administration. A dosage form contains a sufficient amount of active compound such that a desirable effect can be obtained when administered to a subject, preferably prior to at least one meal a day, more preferably prior to each main meal, such as three times a day, during the course of 1 or more days.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging having from 7 to 21 dosage units, or multipla thereof, thereby having dosage units for one week of administration or several weeks of administration.

In one embodiment the medical packaging is for administration once daily in a week, and comprises 7 dosage units, in another embodiment the medical packaging is for administration twice daily, and comprises 14 dosage units. In yet another more preferred embodiment the medical packaging is for administration three times daily, and comprises 21 dosage units.

The dosage units are as defined above, i.e. a dosage unit preferably comprises an amount of the GHS-R1A ligand or a salt thereof equivalent to from 0.3 μg to 600 mg ghrelin, such as of from 2.0 μg to 200 mg ghrelin, such as from 5.0 μg to 100 mg ghrelin, such as from 10 μg to 50 mg ghrelin, such as from 10 μg to 5 mg ghrelin, such as from 10 μg to 1.0 mg ghrelin.

The medical packaging may be in any suitable form for parenteral, in particular subcutaneous administration. In a preferred embodiment the packaging is in the form of a cartridge, such as a cartridge for an injection pen, the injection pen being such as an injection pen known from insulin treatment.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition. In particular said instructions may include instructions referring to administration of said pharmaceutical composition either during a meal, or preferably at the most 45 minutes prior to a meal, such as at the most 30 minutes prior to a meal, such as at the most 25 minutes prior to a meal, such as at the most 20 minutes prior to a meal, such as at the most 15 minutes prior to a meal, such as at the most 10 minutes prior to a meal, such as at the most 5 minutes prior to a meal.

### DESCRIPTION OF FIGURES

FIG. 1: examples of preferred synthetic amino acids for use in the present invention.

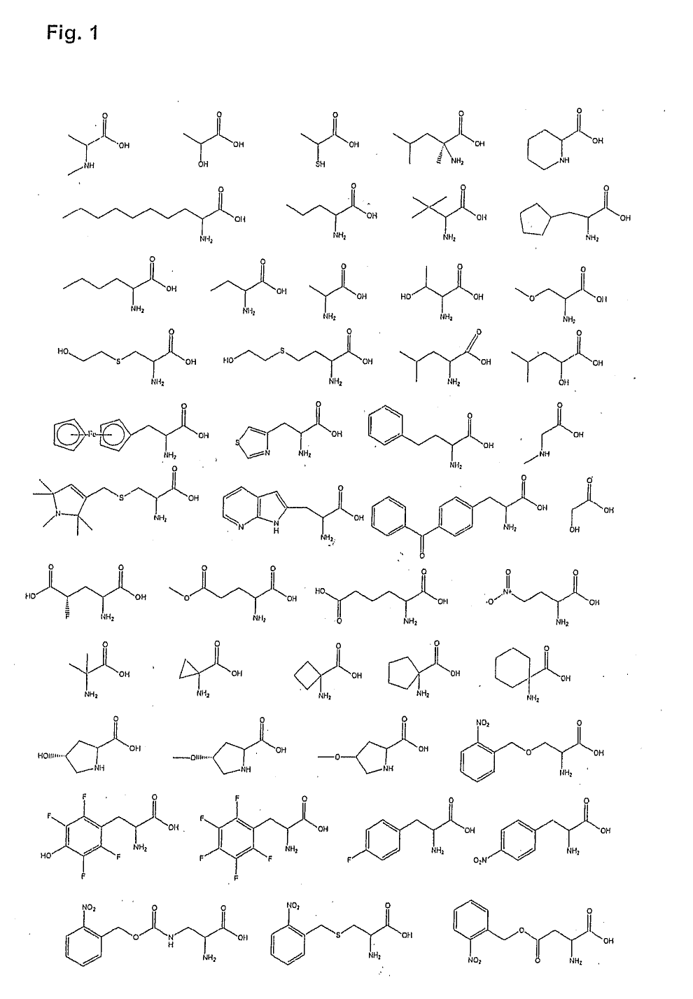

FIG. 2: Total weight gain of rats treated with the compounds of the present invention (see Example 6 for details). The lower part shows the results as box and whiskers plots, where the box extends from the 25^{th} percentile to the 75^{th} percentile, with a line at the median. The whiskers indicate the highest and lowest values.

FIG. 3: Weight of subcutaneous fat pads of rats treated with the compounds of the present invention (see Example 6 for details). Ghrelin and GTP-5 (high dose) induced a significant increase of epididymal fat depots. Ghrelin, GTP-5 (high dose) and GTP-6 (both doses) induced a significant increase of subcutaneous fat depots.

### EXAMPLES

### Example 1

### Competition Binding Assays

Transfected COS-7 cells were transferred to culture plates one day after transfection at a density of 1×10⁵ cells per well aiming at 5-8% binding of the radioactive ligand. Two days after transfection competition binding experiments were performed for 3 hours at 4° C. using 25 pM of ¹²⁵I-ghrelin (Amersham, Little Chalfont, UK). Binding assays were performed in 0.5 ml of a 50 mM Hepes buffer, pH 7.4, supplemented with 1 mM CaCl₂, 5 mM MgCl₂, and 0.1% (w/v) bovine serum albumin, 40 microgram/ml bacitracin. Non-specific binding was determined as the binding in the presence of 1 micromole of unlabeled ghrelin. Cells were washed twice in 0.5 ml of ice-cold buffer and 0.5-1 ml of lysis buffer (8 M Urea, 2% NP40 in 3 M acetic acid) was added and the bound radioactivity was counted. Determinations were made in duplicate. Initial experiments showed that steady state binding was reached with the radioactive ligand under these conditions.

### Example 2

### Rat Pituitary Cell Assay

Sprague-Dawley male albino rats (250≠25 grams) are housed in group cages (four to eight animals/cage) and placed in rooms with 12 hour light cycle. The room temperature is kept at 19-24° C. All media can be obtained from Gibco, trypsin from Worthington, BSA, DNase, T³ and dexamethasone from Sigma (St Louis, USA). The rats are decapitated and the pituitaries dissected. The neurointermediate lobes are removed and the remaining tissue is immediately placed in ice-cold isolation buffer (Gey's medium supplemented with 0.25% D-glucose, 2% non-essential amino acids and 1% BSA, pH 7.3). The tissue is cut into small pieces and transferred to isolation buffer supplemented with 3.8 mg/ml, trypsin and 330 μg/ml DNase. This mixture is incubated at 70 rotations/min for 35 min at 37° C. in a 95/5% atmosphere of O₂/CO₂. The tissue is then washed three times in the above buffer. Using a standard Pasteur pipet, the tissue is then aspirated into single cells. After dispersion, cells are filtered through a nylon filter (160 μm) to remove undigested tissue. The cell suspension is washed 3 times with isolation buffer supplemented with trypsin inhibitor (0.75 mg/ml) and finally resuspended in culture medium; DMEM supplemented with 25 mM HEPES. 4 mM glutamine, 0.075% sodium bicarbonate, 0.1% non-essential amino acid, 2.5% FCS, 3% horse serum, 10% fresh rat serum, 1 nM T³ and 40 μg/L dexamethasone, pH 7.3, to a density of 2×10⁵ cells/ml. The cells are seeded into microtiter plates (Nunc, Roskilde, Denmark), 200 μl/well, and cultured for 3 days at 37° C. and 8% CO².

Following the culture period the cells are washed twice with stimulation buffer (HBSS supplemented with 1% BSA, 0.25% D-glucose and 25 mM HEPES, pH 7.3) and pre-incubated for 1 hour at 37° C. and 5% CO². The buffer is exchanged with new stimulation buffer (37° C.). Test compound solution is added and the plates are incubated for 15 min at 37° C. and 5% CO₂. The medium is decanted and analysed for released GH. All incubations with GHRH are done with human GHRH (1-29NH₂).

### GH-Assay

The rat GH is measured by an in-house developed competitive radio immuno assay (RIA) using I-labelled rat GH, rabbit antibody against rat GH and scintillation proximity assay particles (SPA-particles, Amersham, Buckinghamshire, UK) coated with antibody against rabbit antibody. The detection limit is 4 ng/ml plasma and the intra and inter assay coefficient of variation (CV) are 9.5% and 6.2%, respectively.

### Example 3

### Functional Tests on the GHS-1a Receptor

Transfections and tissue culture—COS-7 cells were grown in Dulbecco's modified Eagle's medium 1885 supplemented with 10% fetal calf serum, 2 mM glutamine and 0.01 mg/ml gentamicin. Cells were transfected using calcium phosphate precipitation method with chloroquine addition as previously described (Holst et al. Mol. Pharm. (1998); 53; 1; p166-175, “Steric hindrance mutagenesis versus alanine scan in mapping of ligand binding sites in the tachykinin NK1 receptor”). For gene dose experiments variable amounts of DNA were used. The amount of cDNA (20 μg/75 cm²) resulting in maximal signaling was the used for dose responds curves. HEK-293 cells were grown in D-MEM, Dulbecco's modified Eagle's medium 31966 with high glucose supplemented with 10% fetal calf serum, 2 mM glutamine and 0.01 mg/ml gentamicin. Cells were transfected with Lipofectamine 2000 (Life Technologies).

Phosphatidylinositol turnover—One day after transfection COS-7 cells were incubated for 24 hours with 5 μCi of [³H]-myo-inositol (Amersham, PT6-271) in 1 ml medium supplemented with 10% fetal calf serum, 2 mM glutamine and 0.01 mg/ml gentamicin per well. Cells were washed twice in buffer, 20 mM HEPES, pH 7.4, supplemented with 140 mM NaCl, 5 mM KCl, 1 mM MgSO₄, 1 mM CaCl₂, 10 mM glucose, 0.05% (w/v) bovine serum; and were incubated in 0.5 ml buffer supplemented with 10 mM LiCl at 37° C. for 30 min. After stimulation with various concentrations of peptide for 45 min at 37° C., cells were extracted with 10% ice-cold perchloric acid followed by incubation on ice for 30 min. The resulting supernatants were neutralized with KOH in HEPES buffer, and the generated [³H]-inositol phosphate was purified on Bio-Rad AG 1-X8 anion-exchange resin as described. Determinations were made in duplicates.

CRE, SRE and NF-κ-B reporter assay. HEK293 cells (30 000 cells/well) seeded in 96-well plates were transiently transfected. In case of the CRE reporter assay the cells were transfected with a mixture of pFA2-CREB and pFR-Luc reporter plasmid (PathDetect CREB trans-Reporting System, Stratagene) or SRE-Luc (PathDetect SRE Cis-Reporting System, Stratagene) and the indicated amounts of receptor DNA. Following transfection cells were maintained in low serum (2.5%) throughout the experiments and were treated with the respective inhibitor of intracellular signaling pathways. One day after transfection, cells were treated with the respective ligands in an assay volume of 100 μl medium for 5 hrs. The assay was terminated by washing the cells twice with PBS and addition of 100 μl luciferase assay reagent (LucLite, Packard). Luminescence was measured in a TopCounter (Top Count NXT™, Packard) for 5 sec. Luminescence values are given as relative light units (RLU).

MAP Kinase assay: COS 7 cells (seeding density 150.000 cells/well) were transfected in the assay plates. Two days after transfection the indicated concentration of ligand were added to assay medium without any serum and incubated for 10 min at 37° C. The reaction were stopped by removing the medium and two washing steps with ice cold PBS. The cells were lysed in sample buffer and separated on SDS/10% PAGE according to Laemmli (“Cleavage of structural proteins during the assembly of the head of bacteriophage T4” Nature vol 227, p680-685). Proteins were transferred onto nitrocellulose and Western blot analysis carried out using 1:5000 dilution of mouse monoclonal antiphopho-ERK1/2 anti-body (Santa Cruz Biotechnology). Total ERK protein was determined using a 1:10000 dilution of anti-ERK antibody (Santa Cruz Biotechnology). Blots were probed anti mouse horseradish peroxidase-conjugated secondary antibodies, visualised using enhanced chemiluminiscence reagent (Amersham Bioscience, New Jersey, US) and quantified by densiometric analysis. ERK1/2 phosphorylation was normalized according to the loading of protein by expressing the data as a ratio of phopho-ERK1/2 over total ERK1/2. Results were expressed as percentage of the value obtained in non stimulated mock transfected cells.

### Example 4

Example format for a two-week efficacy study by sub-cutaneous (s.c.)
route in rats

| | |
|---|---|
| Objective | To establish daily s.c. doses that effectively |
| | increases body weight and total body fat |
| | content as well as increases food consumption. |
| Species/Strain | Rat (Sprague-Dawley, Crl CD ® (SD) IGS BR). |
| Number of animals | 8 males per group. |
| Formulation | Suspension/Solution (to be specified). |
| Treatment groups | Group 1: control (Vehicle). |
| | Group 2: positive control (ghrelin). |
| | Group 3 to 5: one of the compounds disclosed |
| | herein (3 different doses). |
| | Group 6 to 8: one of the compounds disclosed |
| | herein (3 different doses). |
| Mortality, clinical | Twice a day. |
| signs | |
| Body weight, | Daily for the first and last two day and |
| Food and Water | otherwise twice a week. |
| consumption | |
| Blood biochemistry | Pre-dose and on days 7 and 14: |
| | low density lipoprotein (LDL) and high density |
| | lipoprotein (HDL), |
| | phospholipids, |
| | triglycerides. |
| | total cholesterol. |
| Administration | Sub-cutaneous, bi-daily (5 mL/kg). |
| Body composition | Days 7 and 14: whole body: estimations of body |
| assessment in vivo | mineral content, lean body mass, fat content and |
| | % fat proportion using Discovery ™-A QDR |
| | series dual energy X-ray densitometer. |
| Terminal sacrifice | At the end of the study, the animals will be |
| | sacrificed. |
| FAT pads dissection | Dissection and weight of epididymal, |
| | subcutaneous and retro-peritoneal fat-pads |
| Macroscopic | Optional. |
| examination | |
| GLP | The study will not be specifically audited by |
| | Quality Assurance Unit. |

(This assay can also be used to assess the effect of ghrelin antagonists, by showing their ability to reduce food-intake and minimize body-weight gains during the study period.)

### Example 5

### Examples of Suitable Formulations for Preparing Pharmaceutical Compositions for Use in the Present Invention

Two different types of liposomes can be made containing dipalmitoyl DL-α-phosphatidylcholine (DPPC) and a mixture of phosphatidylcholin and cholesterol (PC/Chol), respectively. The liposomes can be prepared by dissolving and mixing the lipids in chloroform. Chloroform will then be removed overnight by rotation evaporation, and the resulting lipid film will first be striped with ethanol (99.9%) and then left overnight in the rotation evaporator. The multilamellar liposomes will then be formed by hydration in HEPES-buffer (10 mM HEPES, 50 mM KCl, 1 mM NaN₃, pH=5.5) for at least 1 h. The hydration temperature was 51° C. (10° C. above the Tₘ of the phospholipids). Subsequently, the liposomes will be sonicated for 30 sec every 10 min during one hour using a tip sonicator. Approx one hundred nanometer unilamellar liposomes will be made from the multilamellar liposomes by extrusion through 100 nm polycarbonate filters, and size measurements performed by dynamic light scattering (DLS) using a Zetasizer 4 (Malvern, UK). Tₘ will be determined by Differential Scanning Caleometry (DSC; MicroCal™ Incorporated). The ligand of the present invention will be added approximately 2 h before the administration of the formulation to the individuals in doses of 60 μg per 500 μL Intralipid 30% may be purchased from the local Pharmacy on the Danish University Hospital Copenhagen (Rigshospitalet). 1000 mL contain: purified soybean oil 300 g, purified egg phospholipids 12 g, glycerol anhydrous 16.7 g, water for injection q.s. ad 1 000 mL. pH is adjusted with sodium hydroxide to pH approximately 7.5.

### Example 6

### Preclinical Study Evaluating the Long-Term Efficacy of Subcutaneous Ghrelin, GTP-5, and GTP-6

### Study Design

48 (Sprauge-Dawley (SD) IGS BR) rats were used in the study. The animals were caged individually and fed a commercial diet, R34, from Lactamin AB, Sweden. They were given free access to water. All animals were allowed an acclimatisation period of a minimum of 7 days prior to the commencement of the experiment. The animals were separated in six groups (n=8) and treated twice daily with subcutaneous injections as follows:
Group 1. Control (NaCl).
Group 2. Positive control Ghrelin 200 μg/kg body weight/injection.
Group 3. GTP-5 50 μg/kg body weight/injection.
Group 4. GTP-5 200 μg/kg body weight/injection.
Group 5. GTP-6 50 μg/kg body weight/injection.
Group 6. GTP-6 200 μg/kg body weight/injection.

### Sequences of Compounds Used:

GTP-5:
27 amino acids long, octanoyl on the serine-3
sidechain (GABA = 4-aminobutyric acid)

(SEQ ID NO: 8)

GABAS(CO-C7 H15)FLSPEHQRVQQRKESKKPPAKLQPR
GTP-6:
28 amino acids long, octanoyl group on serine-3
sidechain (bA = beta-alanine)

(SEQ ID NO: 9)

bASS(CO-C7 H15)FLSPEHQRVQQRKESKKPPAKLQPR

The weight of the animals, and their food and water were recorded daily. The animals were checked twice daily for change in food intake, activity and fur quality as signs of a change in general health status.

Pre-dose, day 7 and 14 the animals were anaesthetized with Isofluran and blood was collected through the orbital plexus. The blood was analysed for clinical chemistry (ASAT, ALAT, bilirubin, albumin, creatinine, LD, GGT, potassium, sodium, glucose, IgF-1, insulin and leptin) and haematology (white blood cells, red blood cells, haemoglobin, platelets and haematokrit). After blood sampling the animals were killed and epididymal, subcutaneous and retroperitoneal fat pads were dissected and weighed.

### Results

All animals gained weight during the study. The ghrelin group gained significantly more weight than the saline group. Furthermore, the GTP-5 (high and low dose) groups and the GTP-6 (high and low dose) groups all showed higher weight gains than saline (Table 1).

TABLE 1
Total weight increase in the animals (g). Mean ± S.D.

| | Weight increase (g) |
|---|---|
| Group 1 Saline | 66.8 ± 14.4 |
| Group 2 Ghrelin | 92.1 ± 9.0 |
| Group 3 GTP-5 50 μg | 75.4 ± 14.3 |
| Group 4 GTP-5 200 μg | 78.2 ± 16.3 |
| Group 5 GTP-6 50 μg | 79.3 ± 10.0 |
| Group 6 GTP-6 200 μg | 77.8 ± 10.2 |

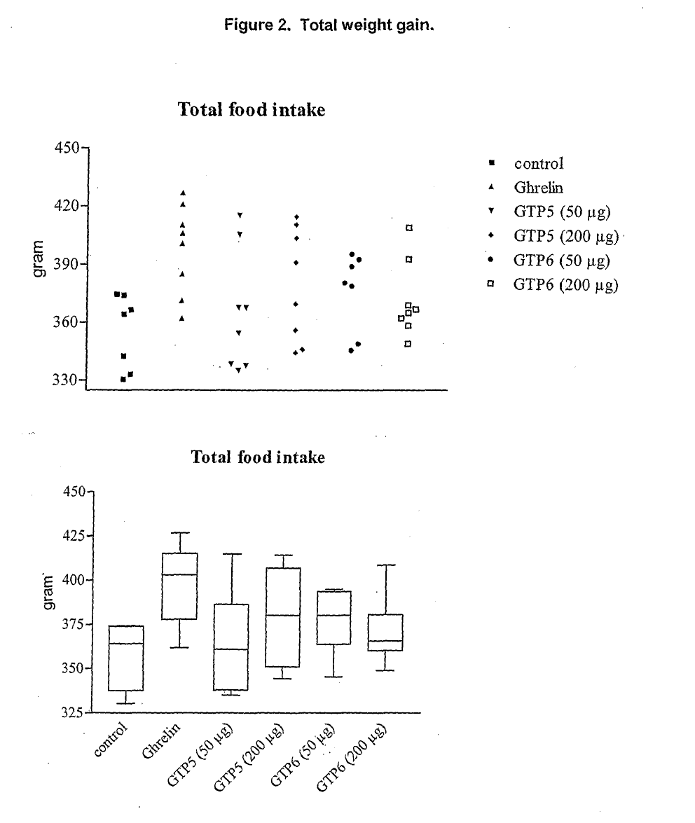

Animals treated with ghrelin showed a higher cumulative food intake in comparison with the control group. Furthermore, the GTP-5 high-dose group and both of the GTP-6 groups tended to eat more than the control group (FIG. 2).

FIG. 2 illustrates the total weight gain. The lower part shows the results as box and whiskers plots, where the box extends from the 25^{th} percentile to the 75^{th} percentile, with a line at the median. The whiskers indicate the highest and lowest values.

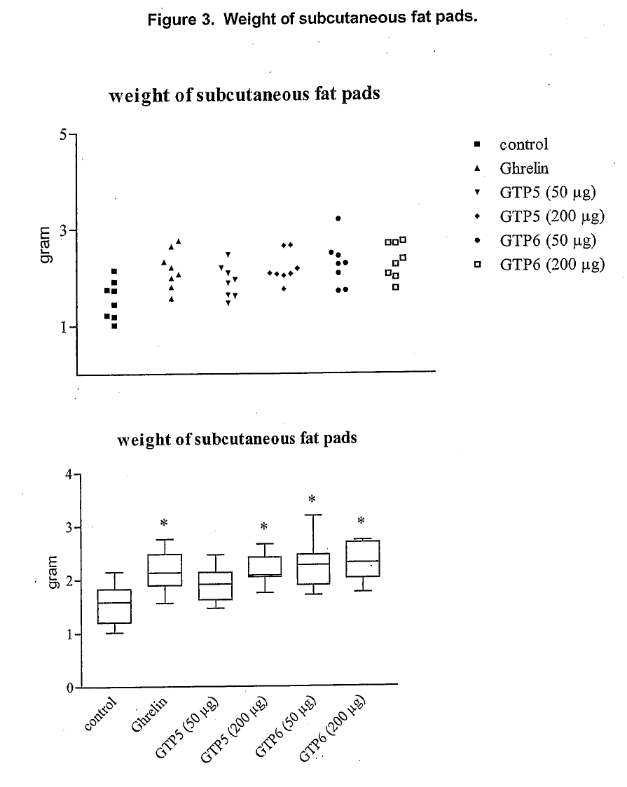

FIG. 3 illustrates the weight of subcutaneous fat pads. Ghrelin and GTP-5 (high dose) induced a significant increase of epididymal fat depots. Ghrelin, GTP-5 (high dose) and GTP-6 (both doses) induced a significant increase of subcutaneous fat depots (FIG. 3).

### Conclusions:

Ghrelin (high dose), GTP-5 (high and low doses) and GTP-6 (high and low doses) were all associated with increases in body weight gain and cumulative food intake.
Ghrelin (high dose), GTP-5 (high dose) and GTP-6 (high and low doses) induced an increase in subcutaneous fat depots.

### Example 7

### Clinical Trial Showing Effect of GHS-R1A Agonists or Antagonists

The GHS-R1a agonist or antagonist according to the present invention (preferably formulated as in Example 5), or a saline control solution, is administered to patients in need thereof by e.g. subcutaneous administration. A preferred dosage is equivalent to 1.0 mg ghrelin administered before each mealtime, such as 20 minutes before a meal.

Efficacy of the compounds can be shown e.g. by examining changes in the individual's BMI, measuring body fat mass, food intake and quality of life questionnaires, as described herein.

### Example 8

### Test for GHS-1R Antagonists

In one embodiment of the present invention, the GHS-R1a ligands is a GHS-R1a antagonist. To test for such antagonism any of the assays mentioned herein, such as in Example 2 or 3, may be used. In the case of the assays mentioned in e.g. Examples 2 or 3, to test for antagonistic effects during stimulation the cells are incubated with either

(i) varying amounts of a GHS-R1a agonist such as ghrelin and the putative antagonist in one or more fixed concentrations or
(ii) with a fixed, submaximal amount of a GHS-R1a agonist and varying amounts of the putative antagonist.

Antagonism can be detected by the ability of the putative antagonist compound to reduce the response to ghrelin in the assay.

### Example 9

### Clinical Protocol for Treatment of Binge Eating Disorder (Bed) with the GHS-R1A Antagonists of the Present Invention

45 subjects that meet the proposed diagnostic criteria/research criteria for BED according to the Diagnostic and Statistical Manual IV (DSM-IV, pub. American Psychiatric Association) are included in the study. The discrete period of time in A1 is a 2-hour period, and the method of determining frequency under item D is counting the number of days on which binges occur.

The study is performed in a double-blinded, placebo-controlled fashion. Subjects are divided into three groups (n=15 in each), groups A, B and C. The subjects are given diaries where they note the type and amount of food ingested and at what time. This initial phase of the study is 4 weeks (“Run-in Phase”), after which the subjects start treatment with one of three regimens, as defined below. The subjects keep diaries where they note the type and amount of food ingested and at what time throughout the treatment phase. The treatment duration is 4 weeks (“Treatment Phase”).

Dosing: The subjects of group A receive subcutaneous placebo injections (NaCl) three times daily (distributed evenly over the hours awake). The subjects of group B receive 5 μg/kg body weight of the GHS-R1a antagonist of the present invention s.c. three times daily and the subjects of group C receive 500 μg/kg body weight of the GHS-R1a antagonist of the present invention s.c. three times daily.

The subjects' diaries are reviewed by the investigators and the number of binge-eating episodes are determined and further the amount of food/calories ingested per binges/event (EDE) is recorded.

The reduction in the frequency of eating disorder events (binge-eating episodes/events), as defined by calculating the number of binge-eating episodes during the 4-week Treatment Phase divided by the number of binge-eating episodes during the Run-in Phase for each subject, and then statistically comparing the result from group B with group A, and the result from group C with group A.

The change in the amount of food ingested during the events is calculated by calculating the number of calories ingested during the binge-eating episodes during the 4-week Treatment Phase divided by the number of calories ingested during the binge-eating episodes during the Run-in Phase for each subject, and then statistically comparing the result from group B with group A, and the result from group C with group A.

### Sequence Listing

(In all cases, polypeptides can be generated as artificial sequences using e.g. synthetic constructs.)

<110> Gastrotech ApS
<120> GHS-R1A ligands
<130> P976DK00
<160> 3
<170> Patentln version 3.1
<210> 1
<211> 28
<212> PRT
<213> Homo sapiens
<220>
<221> MOD_RES
<222> (3) . . . (3)
<223> Amino acid in position 3 is modified with a
fatty acid
<400> 1
SEQ ID NO: 1:
Gly Ser Ser Phe Leu Ser Pro Glu His Gln Arg Val
Gln Gln Arg Lys Glu Ser Lys Lys Pro Pro Ala Lys
Leu Gln Pro Arg
<210> 2
<211> 27
<212> PRT
<213> Homo sapiens
<220>
<221> MOD_RES
<222> (3) . . . (3)
<223> Amino acid in position 3 is modified with a
fatty acid
<400> 2
SEQ ID NO: 2
Gly Ser Ser Phe Leu Ser Pro Glu His Gln Arg Val
Gln Arg Lys Glu Ser Lys Lys Pro Pro Ala Lys Leu
Gln Pro Arg
<210> 3
<211> 28
<212> PRT
<213> Rattus rattus
<220>
<221> MOD_RES
<222> (3) . . . (3)
<223> Amino acid in position 3 is modified with a
fatty acid
<400> 3
SEQ ID NO: 3:
Gly Ser Ser Phe Leu Ser Pro Glu His Gln Lys Ala
Gln Gln Arg Lys Glu Ser Lys Lys Pro Pro Ala Lys
Leu Gln Pro Arg
SEQ ID NO: 4:
28 amino acids long; octanoyl group on the
serine-26 sidechain (acylation)
dRdPdQdLdKdAdPdPdKdKdSdEdKdRdQdQdVdRdQdHdEdPdSdLd
FdS(CO-C7H15)dSG
SEQ ID NO: 5:
28 amino acids long, (PS = phosphatidyl serine)
GS(PS)FLSPEHQRVQQRKESKKPPAKLQPR
SEQ ID NO: 6:
28 amino acids long, (Dec = decenoic acid)
GS(Dec)FLSPEHQRVQQRKESKKPPAKLQPR
SEQ ID NO: 7:
30 amino acids long, octanoyl group on the
serine-3 sidechain
GSS(CO-C7 H15)FLSPEHQRVQQRKESKKPPAKLQPRGG
SEQ ID NO: 8:
27 amino acids long, octanoyl on the serine-3
sidechain (GABA = 4-aminobutyric acid)
GABAS(CO-C7 H15)FLSPEHQRVQQRKESKKPPAKLQPR
SEQ ID NO: 9:
28 amino acids long, octanoyl group on serine-3
sidechain (bA = beta-alanine)
bASS(CO-C7 H15)FLSPEHQRVQQRKESKKPPAKLQPR
SEQ ID NO: 10:
5 amino acids long, octanoyl on d-serine-3
sidechain
dLdFdS(CO-C7 H15)dSG-NH2
SEQ ID NO: 11:
28 amino acids long, myristoyl on the serine-3
sidechain
GSS(CO-C13-H15)FLSPEHQRVQQRKESKKPPAKLQPR
SEQ ID NO: 12:
28 amino acids long, farnesyl on the serine-3
sidechain
GSS(C15-H25)FLSPEHQRVQQRKESKKPPAKLQPR
SEQ ID NO: 13:
28 amino acids long, cholesterol on the serine-3
sidechain
GSS(cholesterol)FLSPEHQRVQQRKESKKPPAKLQPR
SEQ ID NO: 14:
7 amino acids long, (Nal = naphthyl-alanine)
D-Lys-Phe-D-Trp-D-Ala-(2′)-Nal-D-His-D-Ala-NH2
SEQ ID NO: 15:
6 amino acids long, (Nal = naphthyl-alanine)
D-Lys-Nal-D-Trp-D-Ala-(2′)-Nal-D-Ala-NH2
SEQ ID NO: 16:
6 amino acids long
D-Lys-Phe-D-Trp-D-Ala-Trp-D-His-NH2
SEQ ID NO: 17:
6 amino acids long
D-Lys-Phe-D-Trp-D-Ala-2-MeTrp-D-His-NH2
SEQ ID NO: 18:
5 amino acids long, (Nal = naphthyl-alanine,
Aib = aminoisobutyric acid)
D-Lys-Phe-Nal-D-His-Aib-NH2
SEQ ID NO: 19:
4 amino acids long, octanoyl on the serine-2
sidechain, (HAA = hydroxy acetic acid)
HAA-SS(CO-C7 H15)FL-NH2
SEQ ID NO: 20:
28 amino acid long, GABA: 4-aminobutyric acid,
octanoyl on serine-3 sidechain
GABASS(CO-C7 H15)FLSPEHQRVQQRKESKKPPAKLQPR
SEQ ID NO: 21:
27 amino acid long, bA: beta-alanine, octanoyl on
serine-2 sidechain
bAS(CO-C7 H15)FLSPEHQRVQQRKESKKPPAKLQPR
SEQ ID NO: 22:
28 amino acid long, GABA: 4-aminobutyric acid,
octanoyl on serine-2 sidechain
GABAS(CO-C7 H15)FLSPEHQRVQQRKESKKPPAKLQPRGABA
SEQ ID NO: 23:
29 amino acid long, bA: beta-alanine, octanoyl on
serine-3 sidechain
bASS(CO-C7 H15)FLSPEHQRVQQRKESKKPPAKLQPRbA
SEQ ID NO: 24:
27 amino acid long, GABA: 4-aminobutyric acid,
octanoyl on serine-2 sidechain
GABAS(CO-C7 H15)FLSPEHQRVQQRKESKKPPAKLQPR-NH2
SEQ ID NO: 25:
28 amino acid long, bA: beta-alanine, octanoyl on
serine-3 sidechain
bASS(CO-C7 H15)FLSPEHQRVQQRKESKKPPAKLQPR-NH2
SEQ ID NO: 26:
27 amino acid long, GABA: 4-aminobutyric acid,
octanoyl on serine-2 sidechain, (CH2-NH): reduced
peptidebond
GABAS(CO-C7 H15)FLSPEHQRVQQRKESKKPPAKLQP(CH2-NH)R
SEQ ID NO: 27:
28 amino acid long, bA: beta-alanine, octanoyl on
serine-3 sidechain, (CH2-NH): reduced peptidebond
bASS(CO-C7 H15)FLSPEHQRVQQRKESKKPPAKLQP(CH2-NH)R
SEQ ID NO: 28:
27 amino acid long, GABA: 4-aminobutyric acid,
octanoyl on serine-2 sidechain
GABAS(CO-C7 H15)FLSPEHQRVQQRKESKKPPAKLQPdR
SEQ ID NO: 29:
28 amino acid long, bA: beta-alanine, octanoyl on
serine-3 sidechain
bASS(CO-C7 H15)FLSPEHQRVQQRKESKKPPAKLQPdR

It should be understood by one skilled in the art that homologous peptide sequences can be substituted for the polypeptide sequences disclosed herein, such as in SEQ ID NO: 4-29. For example, one may substitute a polypeptide sequence with at least 60% sequence identity to that disclosed herein, such as a polypeptide sequence with at least 65% sequence identity to that disclosed herein, such as a polypeptide sequence with at least 70% sequence identity to that disclosed herein, such as a polypeptide sequence with at least 75% sequence identity to that disclosed herein, such as a polypeptide sequence with at least 80% sequence identity to that disclosed herein, such as a polypeptide sequence with at least 85% sequence identity to that disclosed herein, such as a polypeptide sequence with at least 87% sequence identity to that disclosed herein, such as a polypeptide sequence with at least 90% sequence identity to that disclosed herein, such as a polypeptide sequence with at least 91% sequence identity to that disclosed herein, such as a polypeptide sequence with at least 92% sequence identity to that disclosed herein, such as a polypeptide sequence with at least 93% sequence identity to that disclosed herein, such as a polypeptide sequence with at least 94% sequence identity to that disclosed herein, such as a polypeptide sequence with at least 95% sequence identity to that disclosed herein, such as a polypeptide sequence with at least 96% sequence identity to that disclosed herein, such as a polypeptide sequence with at least 97% sequence identity to that disclosed herein, such as a polypeptide sequence with at least 98% sequence identity to that disclosed herein, such as a polypeptide sequence with at least 99% sequence identity to that disclosed herein. In any case, any of the assays disclosed herein can be used to demonstrate that the homologous polypeptide still affords the desired biological activity (GHS-R1A receptor agonist or antagonist activity)

1. A GHS-R1A ligand compound or a pharmaceutically acceptable salt thereof
wherein the GHS-R1A ligand compound has a structure defined by formula I′
Z²-(X³)ₙ-(X²)ₘ(X¹)ₘZ³-Z¹
wherein Z¹ is an optionally present protecting group,
each X¹ is an amino acid independently selected from naturally occurring and synthetic amino acids,
X² is an anchor group, selected from naturally occurring and synthetic amino acids, said amino acid being modified,
each X³ is independently selected from an amino acid, wherein said amino acid is selected from naturally occurring and synthetic amino acids, with the proviso that at least one (X³) is a D-amino acid,
Z² is an optionally present protecting group,
Z³ is an optionally present linker or C-terminal group,
m is 0 or an integer in the range of 1-3
n is 0 or an integer in the range of 1-35,
and wherein both n and m cannot be 0.

2. The compound according to claim 1, wherein Z³ is selected from the groups
consisting of:
a) βAla or
b) X¹-βAla or
c) GABA or
d) X¹-GABA or
e) -X¹-Aminopentanoyl or
f) hydroxy acetic acid (HAA) or
g) X¹-HAA or
h) a compound with formula B, shown below: 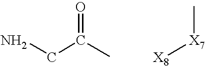
wherein X7 is a spacer with a length of 1-8 chemical bonds, and X8 is a hydrogen bond donor;
wherein m is 2.

3. The compound according claim 1, wherein X² is selected from the group consisting of: modified Ser, modified Cys and modified Lys.

4. The compound according to claim 1, wherein the GHS-R1A ligand compound is selected from the group consisting of:
a compound of formula II′ Z²-(X³)ₙ-(X²)-(X¹)ₘ₋₁-Gly-Z¹,
a compound of formula III′ Z²-(X³)ₙ-(X²)-D-Ser-Gly-Z¹,
a compound of formula IV′ Z²-(X³)ₙ-(X²)-D-Ser-Gly-Z¹,
a compound of formula V′ Z²-(X³)ₙ-D-Ser-Z³-Z¹.

5. The compound according to claim 4, wherein the GHS-R1A ligand compound has formula III′.

6. The compound according to claim 1, comprising a structure having the formula VI′: 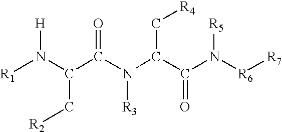
wherein:
R₁ is an alcohol, ether, hydrocarbon, hydrazine, peptide or peptidomimetic moiety,
R₂ is an aromatic moiety,
R₃ is H or CH₃,
R₄ is an aromatic, hydrophobic or amphiphilic moiety,
R₅ is H or CH₃,
R₆ is a spacer with length of 1-8 chemical bonds, and
R₇ is a hydrogen bond donor.

7. The compound according to claim 1, wherein (X³)ₙ comprises a sequence selected from one or more of the sequences shown below:
(SEQ ID NO: 53)
D-Gln D-His D-Glu D-Pro D-Ser D-Leu D-Phe
(SEQ ID NO: 54)
D-His D-Glu D-Pro D-Ser D-Leu D-Phe
(SEQ ID NO: 55)
D-Glu D-Pro D-Ser D-Leu D-Phe
(SEQ ID NO: 56)
D-Pro D-Ser D-Leu D-Phe
(SEQ ID NO: 57)
D-Ser D-Leu D-Phe
D-Leu D-Phe
D-Phe

8. The compound according to claim 1, wherein n is an integer in the range of 1-25.

9. The compound according to claim 1, wherein (X³)ₙ is selected from one or more of the sequences shown below:
(SEQ ID NO: 58)
D-Arg D-Pro D-Gln D-Leu D-Lys D-Ala D-Pro D-Pro
D-Lys D-Lys D-Ser D-Glu D-Lys D-Arg D-Gln D-Gln
D-Val D-Arg D-Gln D-His D-Glu D-Pro D-Ser D-Leu
D-Phe
(SEQ ID NO: 59)
D-Pro D-Gln D-Leu D-Lys D-Ala D-Pro D-Pro D-Lys
D-Lys D-Ser D-Glu D-Lys D-Arg D-Gln D-Gln D-Val
D-Arg D-Gln D-His D-Glu D-Pro D-Ser D-Leu D-Phe
(SEQ ID NO: 60)
D-Gln D-Leu D-Lys D-Ala D-Pro D-Pro D-Lys D-Lys
D-Ser D-Glu D-Lys D-Arg D-Gln D-Gln D-Val D-Arg
D-Gln D-His D-Glu D-Pro D-Ser D-Leu D-Phe
(SEQ ID NO: 61)
D-Leu D-Lys D-Ala D-Pro D-Pro D-Lys D-Lys D-Ser
D-Glu D-Lys D-Arg D-Gln D-Gln D-Val D-Arg D-Gln
D-His D-Glu D-Pro D-Ser D-Leu D-Phe
(SEQ ID NO: 62)
D-Lys D-Ala D-Pro D-Pro D-Lys D-Lys D-Ser D-Glu
D-Lys D-Arg D-Gln D-Gln D-Val D-Arg D-Gln D-His
D-Glu D-Pro D-Ser D-Leu D-Phe
(SEQ ID NO: 63)
D-Arg D-Pro D-Gln D-Leu D-Lys D-Ala D-Pro D-Pro
D-Lys D-Lys D-Ser D-Glu D-Lys D-Arg D-Gln D-Gln
D-Val D-Arg D-Gln D-His D-Glu D-Pro D-Ser D-Leu
D-Phe
(SEQ ID NO: 64)
D-Pro D-Gln D-Leu D-Lys D-Ala D-Pro D-Pro D-Lys
D-Lys D-Ser D-Glu D-Lys D-Arg D-Gln D-Gln D-Val
D-Arg D-Gln D-His D-Glu D-Pro D-Ser D-Leu D-Phe
(SEQ ID NO: 65)
D-Gln D-Leu D-Lys D-Ala D-Pro D-Pro D-Lys D-Lys
D-Ser D-Glu D-Lys D-Arg D-Gln D-Gln D-Val D-Arg
D-Gln D-His D-Glu D-Pro D-Ser D-Leu D-Phe
(SEQ ID NO: 66)
D-Leu D-Lys D-Ala D-Pro D-Pro D-Lys D-Lys D-Ser
D-Glu D-Lys D-Arg D-Gln D-Gln D-Val D-Arg D-Gln
D-His D-Glu D-Pro D-Ser D-Leu D-Phe
(SEQ ID NO: 67)
D-Lys D-Ala D-Pro D-Pro D-Lys D-Lys D-Ser D-Glu
D-Lys D-Arg D-Gln D-Gln D-Val D-Arg D-Gln D-His
D-Glu D-Pro D-Ser D-Leu D-Phe
(SEQ ID NO: 68)
D-Ala D-Pro D-Pro D-Lys D-Lys D-Ser D-Glu D-Lys
D-Arg D-Gln D-Gln D-Val D-Arg D-Gln D-His D-Glu
D-Pro D-Ser D-Leu D-Phe
(SEQ ID NO: 69)
D-Pro D-Pro D-Lys D-Lys D-Ser D-Gln D-Lys D-Arg
D-Gln D-Gln D-Val D-Arg D-Gln D-His D-Glu D-Pro
D-Ser D-Leu D-Phe
(SEQ ID NO: 70)
D-Pro D-Lys D-Lys D-Ser D-Glu D-Lys D-Arg D-Gln
D-Gln D-Val D-Arg D-Gln D-His D-Gln D-Pro D-Ser
D-Leu D-Phe
(SEQ ID NO: 71)
D-Lys D-Lys D-Ser D-Glu D-Lys D-Arg D-Gln D-Gln
D-Val D-Arg D-Gln D-His D-Glu D-Pro D-Ser D-Leu
D-Phe
(SEQ ID NO: 72)
D-Lys D-Ser D-Glu D-Lys D-Arg D-Gln D-Gln D-Val
D-Arg D-Gln D-His D-Glu D-Pro D-Ser D-Leu D-Phe
(SEQ ID NO: 73)
D-Ser D-Glu D-Lys D-Arg D-Gln D-Gln D-Val D-Arg
D-Gln D-His D-Glu D-Pro D-Ser D-Leu D-Phe
(SEQ ID NO: 74)
D-Glu D-Lys D-Arg D-Gln D-Gln D-Val D-Arg D-Gln
D-His D-Glu D-Pro D-Ser D-Leu D-Phe
(SEQ ID NO: 75)
D-Lys D-Arg D-Gln D-Gln D-Val D-Arg D-Gln D-His
D-Glu D-Pro D-Ser D-Leu D-Phe
(SEQ ID NO: 76)
D-Arg D-Gln D-Gln D-Val D-Arg D-Gln D-His D-Glu
D-Pro D-Ser D-Leu D-Phe
(SEQ ID NO: 77)
D-Gln D-Gln D-Val D-Arg D-Gln D-His D-Glu D-Pro
D-Ser D-Leu D-Phe
(SEQ ID NO: 78)
D-Gln D-Val D-Arg D-Gln D-His D-Glu D-Pro D-Ser
D-Leu D-Phe
(SEQ ID NO: 79)
D-Val D-Arg D-Gln D-His D-Glu D-Pro D-Ser D-Leu
D-Phe
(SEQ ID NO: 80)
D-Arg D-Gln D-His D-Glu D-Pro D-Ser D-Leu D-Phe
(SEQ ID NO: 53)
D-Gln D-His D-Glu D-Pro D-Ser D-Leu D-Phe
(SEQ ID NO: 54)
D-His D-Glu D-Pro D-Ser D-Leu D-Phe
(SEQ ID NO: 55)
D-Glu D-Pro D-Ser D-Leu D-Phe
(SEQ ID NO: 56)
D-Pro D-Ser D-Leu D-Phe
(SEQ ID NO: 57)
D-Ser D-Leu D-Phe
D-Leu D-Phe
D-Phe

10. The compound according to claim 1, wherein the anchor group is selected from a C1-C35 acyl group.

11. The compound according to claim 1, wherein the anchor group is selected from the group consisting of: C7 acyl group, C8 acyl group, C9 acyl group, C10 acyl group, C11 acyl group and C12 acyl group.

12. The compound according to claim 1, wherein the anchor group is selected from the group consisting of: C8 acyl group and C10 acyl group.

13. The compound according to claim 1, wherein the anchor group is selected from the group consisting of: C7 acyl group, C9 acyl group, and C11 acyl group.

14. The compound according to claim 1, wherein the anchor group is selected from the group consisting of:
(a) a glycerophospholipid
(b) a sterol moiety
(c) a sphingolipid moiety
(d) a ceramide or an analogue thereof
(e) an isoprenoid pyrophosphate
(f) a glycosyl-phosphatidylinositol (GPI) anchor and
(g) a phosphatidyl serine, or analogue thereof;

15. The compound according to claim 1, wherein the anchor group selected from any of the following:
a glycerophosphate with the following structure: 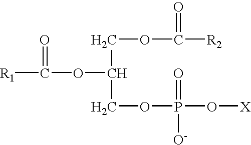
Wherein R1 and R2 represents a saturated or unsaturated hydrocarbon chain, and X represents the amino acid moiety,
a sterol,
a sphingolipid,
a ceramide or an analogue thereof, comprising the following structure: 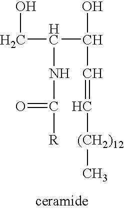
wherein R denotes any hydrocarbon chain (C1-C30), saturated or unsaturated,
an isoprenoid pyrophosphate,
a Glycosyl-phosphatidylinositol (GPI) anchor,
phosphatidyl serine or an analogue thereof,
decenoic acid, or a variant thereof,
8-nonenoic acid (L or D form), or
BenzoTrp (L or D form).

16. The compound according to claim 1, wherein said compound has a sequence according to SEQ ID NO: 4.

17. The compound according to claim 1, wherein said compound has a sequence according to SEQ ID NO: 10.

18-111. (canceled)

112. A GHS-R1A ligand compound or a pharmaceutically acceptable salt thereof,
wherein the GHS-R1A ligand compound has a structure defined by formula I′″
Z¹-R¹-(X²)-(X³)ₙ-Z², wherein
Z¹ is an optionally present protecting group;
R¹ is selected from the group consisting of:
a) βAla-,
b) βAla-X1-,
c) GABA-,
d) GABA-X1-
e) Aminopentanoyl-X1,
f) hydroxy acetic acid (HAA)-,
g) HAA-X1- and
h) a compound with formula B, shown below: 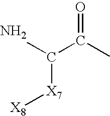
wherein X7 is a spacer with a length of 1-8 chemical bonds, and X8 is a hydrogen bond donor;
X¹ is an amino acid selected from the group consisting of naturally occurring and synthetic amino acids;
X² is an anchor group, which is an amino acid selected from the group consisting of naturally occurring and synthetic amino acids, said amino acid comprising a bulky group;
each X³ is an independently selected amino acid, wherein said amino acid is selected from naturally occurring and synthetic amino acids,
wherein X³ optionally may be an anchor group,
Z² is an optionally present protecting group,
n is 0 or an integer in the range of 1-35.

113. The GHS-R1A ligand compound according to claim 112, wherein R¹ is selected from the group consisting of:
a) βAla-
b) βAla-X¹-
c) GABA-
d) GABA-X¹-
e) hydroxy acetic acid (HAA)-
f) HAA-X¹- and
g) a compound with formula B, shown below:

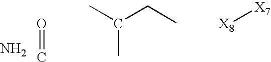

114. The compound according to claim 13, wherein X² is selected from the group consisting of: modified Ser, modified Cys and modified Lys.

115. The compound according to claim 113, wherein X² is selected from the group consisting of:
a) decenoic acid (L or D form),
b) 5-hexenoic acid (L or D form),
c) 6-heptenoic acid (L or D form),
d) 7-octenoic acid (L or D form),
e) 8-nonenoic acid (L or D form),
f) Ala-3-cp (L or D form),
g) Ala-3-cb (L or D form),
h) Phe-4-Me (L or D form),
i) Phe-4-Et (L or D form),
j) Phe-4-iPr (L or D form),
k) Phe-4-Ph (L or D form),
l) Beta-MeTrp (L or D form),
m) Ala[3-(3-Quinolinyl)] (L or D form),
n) Ala[3-(2-benzimidazoyl)] (L or D form),
o) BenzoTrp (L or D form),
p) 7-AzaTrp (L or D form) and
q) Trp(5-NH2) (L or D form);

116. The compound according to claim 112, wherein the GHS-R1A ligand compound is selected from the group consisting of:
Z¹-βAla-(X²)-(X³)ₙ-Z², formula II′″
Z¹-βAla-Ser-(X²)-(X³)ₙ-Z², formula III′″
Z¹-GABA-(X²)-(X³)ₙ-Z², formula IV′″
Z¹-GABA-Ser-(X²)-(X³)ₙ-Z², formula V′″
Z¹-Aminopentanoyl-Ser-(X²)-(X³)ₙ-Z², formula VII′″
Z¹-HAA-Ser-(X²)-(X³)ₙ-Z², formula VIII′″
Z¹-HAA-(X²)-(X³)ₙ-Z² formula IX′″
wherein Z¹ and Z² are optional protecting groups.

117. The compound according to any of claims 112-116, wherein n is an integer in the range of 1-25.

118. The compound according claim 112, wherein (X³)ₙ is as defined in claims 7-8.

119. The compound according to claim 112, wherein the anchor group is selected from a C1-C35 acyl group.

120. The compound according to claim 112, wherein said compound has SEQ ID NO: 8.

121. The compound according to claim 112, wherein said compound has SEQ ID NO: 9.

122. The compound according to claim 112, wherein said compound consists of any of the sequences with SEQ ID NO: 19-29.

123. A pharmaceutical composition comprising the GHS-R1A ligand compound as defined in claim 1 or a pharmaceutically acceptable salt thereof and pharmaceutically acceptable carriers, vehicles and/or excipients.

124. The pharmaceutical composition according to claim 123, wherein said composition further comprises transport molecules, such as liposomes, micelles, iscoms, and/or microspheres.

125. A medical packaging comprising one or more dosage units of the pharmaceutical composition as defined in claim 123.

126-131. (canceled)

132. A method of treatment of a growth hormone secretagogue receptor 1A (GHS-R1A) associated disorder comprising administering a therapeutically effective amount of a GHS-R1A ligand compound according to claim 1 to an individual in need thereof.

133. The method of claim 132 wherein the compound is administered subcutaneously.

134. A method of treatment of a pathological condition treatable with a GHS-R1A ligand comprising administering a therapeutically effective amount of a GHS-R1A ligand compound according to claim 1 to an individual in need thereof.

135. The method of claim 132 comprising treatment of cachexia and/or malnutrition.

136. A method of simulating appetite, food intake and/or weight gain, and/or increasing body fat mass and/or lean body mass, comprising administering a therapeutically effective amount of a GHS-R1A ligand compound according to claim 1 to an individual in need thereof.

137. A method of maintaining metabolic homeostasis comprising administering a therapeutically effective amount of a GHS-R1A ligand compound according to claim 1 to an individual in need thereof.
